(19) United States
(12) Patent Application Publication
(10) Pub. No.: US 2026/0283490 A1
(43) Pub. Date: Sep. 24, 2026

(54) Systems And Methods For Measuring Cardiac Output

(71) Applicant: Becton, Dickinson and Company, Franklin Lakes, NJ (US)

(72) Inventors: Monera Amani Kamal, Orange, CA (US); Zhongping Jian, Irvine, CA (US); Feras Al Hatib, Irvine, CA (US); Kendra Nicole Washington, Irvine, CA (US); Minh Thieu Huynh, Cerritos, CA (US)

(21) Appl. No.: 19/474,452

(22) PCT Filed: Apr. 10, 2024
(86) PCT No.: PCT/US2024/023853

§ 371 Date: Oct. 10, 2025

Related U.S. Application Data
(60) Provisional application No. 63/495,283, filed on Apr. 10, 2023.

(51) Int. Cl. A61B 5/029; A61B 5/00; A61B 5/0215; A61B 5/022; A61B 5/028

(52) U.S. Cl. CPC A61B 5/029; A61B 5/0215; A61B 5/022; A61B 5/028; A61B 5/7278

(57) ABSTRACT

Systems and methods for monitoring hemodynamic parameters are described. The systems and methods can combine hemodynamic parameters to yield enhanced parameters.

### CROSS REFERENCE TO RELATED PATENTS

The current application claims the benefit of and priority to U.S. Provisional Patent Application No. 63/495,283 entitled “Systems and Methods for Measuring Cardiac Output” filed Apr. 10, 2023, the disclosure of which is hereby incorporated by reference in its entirety for all purposes.

### BACKGROUND

Cardiac output (CO) provides an indication of the volume of blood being pumped by the heart of a patient at any given time. There are numerous techniques for determining cardiac output, including arterial pressure cardiac output (APCO) style algorithms, and the continuous cardiac output (CCO) and injectate cardiac output (ICO) thermal dilution style algorithms. APCO style algorithms generally utilize hemodynamic data acquired using a peripheral hemodynamic sensor such as a peripheral arterial line (PAL) or a blood pressure cuff. CCO and ICO thermal dilution style algorithms utilize hemodynamic data acquired using a central hemodynamic sensor such as a pulmonary artery catheter (PAC).

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some implementations, a method is for hemodynamic monitoring of a patient to provide a combined cardiac output measurement from two hemodynamic sensors. The method comprises sensing, using a peripheral arterial pressure sensor, a peripheral arterial pressure signal of a patient. The method comprises deriving, using a computational processing system and an arterial pressure-based cardiac output (APCO) algorithm, an APCO of the patient based upon the peripheral arterial pressure signal. The method comprises sensing, using a central artery catheter sensor, a central blood flow signal of the patient. The method comprises deriving, using the computational processing system and a thermodilution based cardiac output algorithm, a thermodilution based cardiac output of the patient based upon the central blood flow signal. The method comprises deriving, using the computational processing system, a time-varying linear scaling equation. The method comprises computing, using the computational processing system and the derived time-varying linear scaling equation, a combined cardiac output. Computing the combined cardiac output comprises applying a scaling factor and an offset factor to the APCO.

In some implementations, the derivation of the time-varying linear scaling equation comprises determining a scaling factor and an offset factor. Determining the scaling factor comprises determining whether the scaling factor is a preset value or a computed value using a temporally weighted APCO and a temporally weighted thermodilution based cardiac output. Determining the offset factor comprises using the scaling factor, the temporally weighted APCO, and the temporally weighted thermodilution based cardiac output.

In some implementations, the APCO is iteratively derived at a first frequency and the thermodilution based cardiac output is iteratively derived at a second frequency.

In some implementations, the combined cardiac output is iteratively computed at the first frequency.

In some implementations, the scaling factor and the offset factor are each iteratively determined at the second frequency.

In some implementations, the time-varying linear scaling equation is:

$${FusedCO}_{n 1} = {A}_{n 2} \times {APCO}_{n 1} + {B}_{n 2}$$

n1 is the first frequency, n2 is the second frequency, A is the scaling factor, B is the offset factor, and FusedCO is the combined cardiac output.

In some implementations, the first frequency is greater than the second frequency.

In some implementations, the scaling factor is determined to be the preset value when the difference between the temporally weighted APCO and the temporally weighted thermodilution based cardiac output is less than a first cardiac output threshold and a first combined cardiac index of the patient is greater than a cardiac index threshold.

In some implementations, the preset value is 1.

In some implementations, the scaling factor is determined to be the computed value when the difference between the temporally weighted APCO and the temporally weighted thermodilution based cardiac output is greater than the first cardiac output threshold.

In some implementations, when the difference between the temporally weighted APCO and the temporally weighted thermodilution based cardiac output is greater than the first cardiac output threshold and less than a second cardiac output threshold, the computed value of the scaling factor is computed as follows:

$${A}_{reduced} = {A}_{preset} \times [ \frac{{S}_{A}}{{S}_{F}} \times ( {CO}_{diff} - {CO}_{lowThresh} ) + ( {CO}_{highThresh} - {CO}_{diff} ) ]$$

A_{reduced} is the computed value of the scaling factor; Aₚᵣₑₛₑₜ is the preset value of the scaling factor; S_{F} is the temporally weighted APCO; S_{A} is the temporally weighted thermodilution based cardiac output; COthresh1 is the first cardiac output threshold; COthresh2 is the second cardiac output threshold; COthresh1<COthresh2; and

COdiff=S_{F}−S_{A}.

In some implementations, when the difference between the temporally weighted APCO and the temporally weighted thermodilution based cardiac output (S_{A}) is greater than or equal to the second cardiac output threshold, the computed value of the scaling factor is computed as follows:

$${A}_{reduced} = {A}_{preset} \times \frac{{S}_{A}}{{S}_{F}}$$

A_{reduced} is the computed value of the scaling factor; Aₚᵣₑₛₑₜ is the preset value of the scaling factor; S_{F} is the temporally weighted APCO and S_{A} is the temporally weighted thermodilution based cardiac output.

In some implementations, the computed value of the scaling factor is further reduced by a first numerical value when an updated combined cardiac index is less than the cardiac index threshold. The updated combined cardiac index is computed using the computed value of the scaling factor of claim 10 or claim 11.

In some implementations, the computed value of the scaling factor is further reduced by a second numerical value when a further updated combined cardiac index is less than the cardiac index threshold. The further updated combined cardiac index is computed using the computed value of the scaling factor of claim 12.

In some implementations, the scaling factor is determined to be the computed value when the first combined cardiac index of the patient is less than the cardiac index threshold. When the first combined cardiac index of the patient is less than the cardiac index threshold, the computed value of the scaling factor is computed by reducing the preset scaling factor by a first numerical factor.

In some implementations, the computed value of the scaling factor is further reduced by a second numerical value when an updated combined cardiac index is less than the cardiac index threshold. The further updated combined cardiac index is computed using the computed value of the scaling factor of claim 14.

In some implementations, the combined cardiac index or the updated combined cardiac index is computed as follows:

$${FusedCI}_{n 1} = \frac{{FusedCO}_{n 1}}{B S A}$$

BSA is a body surface area of the patient and FusedCI is the combined cardiac index or the updated combined cardiac index.

In some implementations, the offset is determined by the following equation:

$$B = \frac{( {S}_{FA} - A \times {S}_{FF} - {S}_{A} + A \times {S}_{F} )}{( {S}_{F} - M - β )}$$

B is the offset, A is the scaling, β is a regularization parameter, where S_{F} is the temporally weighted APCO, S_{A} is the temporally weighted CCO, S_{FA} is the temporally weighted inner product of APCO and CCO, S_{FF} is the temporally weighted inner product of APCO with itself, and M is the temporally weighted counter.

In some implementations, β≥0.

In some implementations, initial values are as follows: S_{F}=0, S_{FF}=0, S_{A}=0, S_{FA}=0, M=0, and γ is a forgetting factor that is >0 and <1.

In some implementations, the method further comprises displaying the combined time varying cardiac output on an electronic visual display.

In some implementations, the method further comprises providing an alert or fault if the combined computed cardiac index of the patient is above a high threshold or below a low threshold.

In some implementations, the method further comprises providing an alert or fault if a trend the combined computed cardiac declines at a rate greater than a declination threshold or ascends at a rate greater than an ascension threshold.

In some implementations, the peripheral arterial pressure sensor is a peripheral arterial line catheter.

In some implementations, the peripheral arterial pressure sensor is a blood pressure cuff.

In some implementations, a system is for hemodynamic monitoring of a patient to provide a combined cardiac output measurement from two hemodynamic sensors. The system comprises a peripheral arterial pressure sensor for sensing a peripheral arterial pressure signal. The system comprises a pulmonary artery catheter for sensing a central blood flow signal. The system comprises a computational processing system in operable connection with the peripheral arterial pressure sensor and the pulmonary artery catheter. The computational processing system comprises a processor system. The computational processing system comprises. a memory system comprising one or more applications. The one or more applications can direct the processor system to derive, using an arterial pressure-based cardiac output (APCO) algorithm, an APCO based upon the peripheral arterial pressure signal. The one or more applications can direct the processor system to derive, using a thermodilution based cardiac output algorithm, a thermodilution based cardiac output based upon the central blood flow signal. The one or more applications can direct the processor system to derive a time-varying linear scaling equation. The derivation of the time-varying linear scaling equation comprises determining a scaling factor and an offset factor. Determining the scaling factor comprises determining whether the scaling factor is a preset value or a computed value using a temporally weighted APCO and a temporally weighted thermodilution based cardiac output. Determining the offset factor comprises using the scaling factor, the temporally weighted APCO, and the temporally weighted thermodilution based cardiac output. The one or more applications can direct the processor system to compute, using the derived time-varying linear scaling equation, a combined cardiac output. Computing the combined cardiac output comprises applying a scaling factor and an offset factor to the APCO.

In some implementations, the APCO is iteratively derived at a first frequency and the thermodilution based cardiac output is iteratively derived at a second frequency.

In some implementations, the combined cardiac output is iteratively computed at the first frequency.

In some implementations, the scaling factor and the offset factor are each iteratively determined at the second frequency.

In some implementations, the time-varying linear scaling equation is:

$${FusedCO}_{n 1} = {A}_{n 2} \times {APCO}_{n 1} + {B}_{n 2}$$

n1 is the first frequency, n2 is the second frequency, A is the scaling factor, B is the offset factor, and FusedCO is the combined cardiac output.

In some implementations, the first frequency is greater than the second frequency.

In some implementations, the scaling factor is determined to be the preset value when the difference between the temporally weighted APCO and the temporally weighted thermodilution based cardiac output is less than a first cardiac output threshold and a first combined cardiac index of the patient is greater than a cardiac index threshold.

In some implementations, the preset value is 1.

In some implementations, the scaling factor is determined to be the computed value when the difference between the temporally weighted APCO and the temporally weighted thermodilution based cardiac output is greater than the first cardiac output threshold.

In some implementations, when the difference between the temporally weighted APCO and the temporally weighted thermodilution based cardiac output is greater than the first cardiac output threshold and less than a second cardiac output threshold, the computed value of the scaling factor is computed as follows:

$${A}_{reduced} = {A}_{preset} \times [ \frac{{S}_{A}}{{S}_{F}} \times ( {CO}_{diff} - {CO}_{lowThresh} ) + ( {CO}_{highThresh} - {CO}_{diff} ) ]$$

A_{reduced} is the computed value of the scaling factor; Aₚᵣₑₛₑₜ is the preset value of the scaling factor; S_{F} is the temporally weighted APCO; S_{A} is the temporally weighted thermodilution based cardiac output; COthresh1 is the first cardiac output threshold; COthresh2 is the second cardiac output threshold; COthresh1<COthresh2; and

COdiff=S_{F}−S_{A}.

In some implementations, when the difference between the temporally weighted APCO and the temporally weighted thermodilution based cardiac output (S_{A}) is greater than or equal to the second cardiac output threshold, the computed value of the scaling factor is computed as follows:

$${A}_{reduced} = {A}_{preset} \times \frac{{S}_{A}}{{S}_{F}}$$

A_{reduced} is the computed value of the scaling factor; Aₚᵣₑₛₑₜ is the preset value of the scaling factor; S_{F} is the temporally weighted APCO and S_{A} is the temporally weighted thermodilution based cardiac output.

In some implementations, the computed value of the scaling factor is further reduced by a first numerical value when an updated combined cardiac index is less than the cardiac index threshold. The updated combined cardiac index is computed using the computed value of the scaling factor of claim 10 or claim 11.

In some implementations, the computed value of the scaling factor is further reduced by a second numerical value when a further updated combined cardiac index is less than the cardiac index threshold. The further updated combined cardiac index is computed using the computed value of the scaling factor of claim 12.

In some implementations, the scaling factor is determined to be the computed value when the first combined cardiac index of the patient is less than the cardiac index threshold. When the first combined cardiac index of the patient is less than the cardiac index threshold, the computed value of the scaling factor is computed by reducing the preset scaling factor by a first numerical factor.

In some implementations, the computed value of the scaling factor is further reduced by a second numerical value when an updated combined cardiac index is less than the cardiac index threshold. The further updated combined cardiac index is computed using the computed value of the scaling factor of claim 14.

In some implementations, the combined cardiac index or the updated combined cardiac index is computed as follows:

$${FusedCI}_{n 1} = \frac{{FusedCO}_{n 1}}{B S A}$$

BSA is a body surface area of the patient and FusedCI is the combined cardiac index or the updated combined cardiac index.

In some implementations, the offset is determined by the following equation:

$$B = \frac{( {S}_{FA} - A \times {S}_{FF} - {S}_{A} + A \times {S}_{F} )}{( {S}_{F} - M - β )}$$

B is the offset, A is the scaling, β is a regularization parameter, where S_{F} is the temporally weighted APCO, S_{A} is the temporally weighted CCO, S_{FA} is the temporally weighted inner product of APCO and CCO, S_{FF} is the temporally weighted inner product of APCO with itself, and M is the temporally weighted counter.

In some implementations, β≥0.

In some implementations, initial values are as follows: S_{F}=0, S_{FF}=0, S_{A}=0, S_{FA}=0, M=0, and γ is a forgetting factor that is >0 and <1.

In some implementations, the one or more applications that can further direct the processor system to display the combined time varying cardiac output on an electronic visual display.

In some implementations, the one or more applications that can further direct the processor system to provide an alert or fault if the combined computed cardiac index of the patient is above a high threshold or below a low threshold.

In some implementations, the one or more applications that can further direct the processor system to provide an alert or fault if a trend the combined computed cardiac declines at a rate greater than a declination threshold or ascends at a rate greater than an ascension threshold.

In some implementations, the peripheral arterial pressure sensor is a peripheral arterial line catheter.

In some implementations, the peripheral arterial pressure sensor is a blood pressure cuff.

In some implementations, a method is for hemodynamic monitoring of a patient to provide a combined cardiac output measurement from two hemodynamic sensors. The method comprises sensing, using a peripheral arterial pressure sensor, a peripheral arterial pressure signal of a patient. The method comprises deriving, using a computational processing system and an arterial pressure-based cardiac output (APCO) algorithm, an APCO of the patient based upon the peripheral arterial pressure signal. The method comprises sensing, using a pulmonary artery catheter, a central blood flow signal of the patient. The method comprises deriving, using the computational processing system and a thermodilution based cardiac output algorithm, a thermodilution based cardiac output of the patient based upon the central blood flow signal. The method comprises deriving, using the computational processing system, a time-varying linear scaling equation. The method comprises computing, using the computational processing system and the derived time-varying linear scaling equation, a combined cardiac output. Computing the combined cardiac output comprises applying a scaling factor and an offset factor to the APCO.

In some implementations, the derivation of the time-varying linear scaling equation comprises setting a scaling factor to a preset value. The derivation of the time-varying linear scaling equation comprises determining an offset factor. Determining the offset factor comprises using the scaling factor, the temporally weighted APCO, and the temporally weighted thermodilution based cardiac output.

In some implementations, the APCO is iteratively derived at a first frequency and the thermodilution based cardiac output is iteratively derived at a second frequency.

In some implementations, the combined cardiac output is iteratively computed at the first frequency.

In some implementations, the scaling factor and the offset factor are each iteratively determined at the second frequency.

In some implementations, the time-varying linear scaling equation is:

$${FusedCO}_{n 1} = {A}_{n 2} \times {APCO}_{n 1} + {B}_{n 2}$$

n1 is the first frequency, n2 is the second frequency, A is the scaling factor, B is the offset factor, and FusedCO is the combined cardiac output.

In some implementations, the first frequency is greater than the second frequency.

In some implementations, the offset is determined by the following equation:

$$B = \frac{( {S}_{FA} - A \times {S}_{FF} - {S}_{A} + A \times {S}_{F} )}{( {S}_{F} - M - β )}$$

B is the offset, A is the scaling, β is a regularization parameter, where S_{F} is the temporally weighted APCO, S_{A} is the temporally weighted CCO, S_{FA} is the temporally weighted inner product of APCO and CCO, S_{FF} is the temporally weighted inner product of APCO with itself, and M is the temporally weighted counter.

In some implementations, β≥0.

In some implementations, initial values are as follows: S_{F}=0, S_{FF}=0, S_{A}=0, S_{FA}=0, M=0, and γ is a forgetting factor that is >0 and <1.

In some implementations, the method further comprises displaying the combined time varying cardiac output on an electronic visual display.

In some implementations, the method further comprises providing an alert or fault if the combined computed cardiac index of the patient is above a high threshold or below a low threshold.

In some implementations, the method further comprises providing an alert or fault if a trend the combined computed cardiac declines at a rate greater than a declination threshold or ascends at a rate greater than an ascension threshold.

In some implementations, the peripheral arterial pressure sensor is a peripheral arterial line catheter.

In some implementations, the peripheral arterial pressure sensor is a blood pressure cuff.

In some implementations, a system is for hemodynamic monitoring of a patient to provide a combined cardiac output measurement from two hemodynamic sensors. The system comprises a peripheral arterial pressure sensor for sensing a peripheral arterial pressure signal. The system comprises a pulmonary artery catheter for sensing a central blood flow signal. The system comprises a computational processing system in operable connection with the peripheral arterial pressure sensor and the pulmonary artery catheter. The computational processing system comprises a processor system. The computational processing system comprises. a memory system comprising one or more applications. The one or more applications can direct the processor system to derive, using an arterial pressure-based cardiac output (APCO) algorithm, an APCO based upon the peripheral arterial pressure signal. The one or more applications can direct the processor system to derive, using a thermodilution based cardiac output algorithm, a thermodilution based cardiac output based upon the central blood flow signal. The one or more applications can direct the processor system to derive a time-varying linear scaling equation. The derivation of the time-varying linear scaling equation comprises setting a scaling factor to a preset value. The derivation of the time-varying linear scaling equation comprises determining an offset factor. Determining the offset factor comprises using the scaling factor, the temporally weighted APCO, and the temporally weighted thermodilution based cardiac output. The one or more applications can direct the processor system to compute, using the derived time-varying linear scaling equation, a combined cardiac output. Computing the combined cardiac output comprises applying a scaling factor and an offset factor to the APCO.

In some implementations, the APCO is iteratively derived at a first frequency and the thermodilution based cardiac output is iteratively derived at a second frequency.

In some implementations, the combined cardiac output is iteratively computed at the first frequency.

In some implementations, the scaling factor and the offset factor are each iteratively determined at the second frequency.

In some implementations, the time-varying linear scaling equation is:

$${FusedCO}_{n 1} = {A}_{n 2} \times {APCO}_{n 1} + {B}_{n 2}$$

n1 is the first frequency, n2 is the second frequency, A is the scaling factor, B is the offset factor, and FusedCO is the combined cardiac output.

In some implementations, the first frequency is greater than the second frequency.

In some implementations, the offset is determined by the following equation:

$$B = \frac{( {S}_{FA} - A \times {S}_{FF} - {S}_{A} + A \times {S}_{F} )}{( {S}_{F} - M - β )}$$

B is the offset, A is the scaling, β is a regularization parameter, where S_{F} is the temporally weighted APCO, S_{A} is the temporally weighted CCO, S_{FA} is the temporally weighted inner product of APCO and CCO, S_{FF} is the temporally weighted inner product of APCO with itself, and M is the temporally weighted counter.

In some implementations, β≥0.

In some implementations, initial values are as follows: S_{F}=0, S_{FF}=0, S_{A}=0, S_{FA}=0, M=0, and γ is a forgetting factor that is >0 and <1.

In some implementations, the one or more applications that can further direct the processor system to display the combined time varying cardiac output on an electronic visual display.

In some implementations, the one or more applications that can further direct the processor system to provide an alert or fault if the combined computed cardiac index of the patient is above a high threshold or below a low threshold.

In some implementations, the one or more applications that can further direct the processor system to provide an alert or fault if a trend the combined computed cardiac declines at a rate greater than a declination threshold or ascends at a rate greater than an ascension threshold.

In some implementations, the peripheral arterial pressure sensor is a peripheral arterial line catheter.

In some implementations, the peripheral arterial pressure sensor is a blood pressure cuff.

### BRIEF DESCRIPTION OF THE DRAWINGS

The description and claims will be more fully understood with reference to the following figures, which are presented as examples of the disclosure and should not be construed as a complete recitation of the scope of the disclosure.

FIG. 1 provides a schematic of an example of a hemodynamic monitoring system for combining a hemodynamic parameter from two data sources.

FIG. 2 provides a schematic of an example of a decision tree for dynamic scaling.

FIG. 3 provides an example of a method for deriving and providing a combined hemodynamic parameter from two data sources.

FIG. 4 provides a schematic of an example of a computational system for combining a hemodynamic parameter from two data sources.

FIG. 5 provides a data graph of simulated CO parameters and combined CO parameters.

### DETAILED DESCRIPTION

The current disclosure details systems, devices, and methods for hemodynamic monitoring. The systems and devices can comprise at least two hemodynamic sensors for use on a patient to be monitored. Hemodynamic measurements provided by the at least two sensors can be combined to yield an enhanced measurement. For instance, in some implementations, the at least two sensors can each sense hemodynamic data (e.g., blood pressure), which can be utilized to derive two cardiac output readings, which can be combined. The systems, devices, and methods described herein can be utilized as an independent monitoring system or utilized in a combination with any other compatible hemodynamic monitoring system.

Cardiac output can be derived from an arterial pressure. Various arterial pressure measurements yielded by arterial pressure sensors can yield different advantages. For instance, peripheral arterial pressures can be sensed by (for example) a blood pressure cuff and a peripheral arterial line (PAL). These peripheral arterial pressures can have rapid sampling to allow for continuous monitoring and timely recognition of rapid changes of hemodynamic parameters, including cardiac output. Alternatively, a central artery catheter sensor (e.g., pulmonary artery catheter) provides centralized hemodynamic monitoring and yields a more accurate continuous CO parameter, but generally yields CO at a slower rate than that of a peripheral sensor. An enhanced cardiac output can be generated by combining cardiac outputs using a peripheral arterial sensor and a central artery catheter (see U.S. application Ser. No. 16/905,196, the disclosure of which is incorporated herein by reference). In some implementations, a time varying linear scaling equation can be utilized to derive a combined cardiac output. In some implementations, the following time varying linear scaling equation is utilized to derive a combined cardiac output:

$${FusedCO}_{n 1} = {A}_{n 2} \times {APCO}_{n 1} + {B}_{n 2} \qquad ( A1 )$$

where FusedCOₙ₁ is the combined cardiac output, APCO is a peripheral arterial pressure cardiac output, A is a scaling factor, B is an offset factor, n1 is the frequency that the peripheral arterial pressure cardiac output is derived, and n2 is the frequency that the central continuous cardiac output is derived.

The frequency that the peripheral arterial pressure cardiac output (n1) is derived can be any iteratively repeated amount of time. The more frequent the peripheral arterial pressure cardiac output is derived, the more data outputs for a combined CO waveform. Higher frequency yields a more current CO waveform but requires greater computation. Thus, a balance between the waveform output and computation can be selected and/or optimized. In some implementations, n1 is between an order of partial seconds to an order of minutes. In some implementations, n1 is between 0.1 seconds and 300 seconds. In various implementations, n1 is about 0.1 seconds, n1 is about 0.5 seconds, n1 is about 1 second, n1 is about 2 seconds, n1 is about 3 seconds, n1 is about 4 seconds, n1 is about 5 seconds, n1 is about 6 seconds, n1 is about 7 seconds, n1 is about 8 seconds, n1 is about 9 seconds, n1 is about 10 seconds, n1 is about 11 seconds, n1 is about 12 seconds, n1 is about 13 seconds, n1 is about 14 seconds, n1 is about 15 seconds, n1 is about 16 seconds, n1 is about 17 seconds, n1 is about 18 seconds, n1 is about 19 seconds, n1 is about 20 seconds, n1 is about 21 seconds, n1 is about 22 seconds, n1 is about 23 seconds, n1 is about 24 seconds, n1 is about 25 seconds, n1 is about 26 seconds, n1 is about 27 seconds, n1 is about 28 seconds, n1 is about 29 seconds, n1 is about 30 seconds, n1 is about 45 seconds, n1 is about 60 seconds, n1 is about 75 seconds, n1 is about 90 seconds, n1 is about 105 seconds, n1 is about 120 seconds, n1 is about 135 seconds, n1 is about 150 seconds, n1 is about 165 seconds, n1 is about 180 seconds, n1 is about 195 seconds, n1 is about 210 seconds, n1 is about 225 seconds, n1 is about 240 seconds, n1 is about 255 seconds, n1 is about 270 seconds, n1 is about 285 seconds, or n1 is about 300 seconds.

The frequency that the central arterial continuous cardiac output (n2) is derived can be any iteratively repeated amount of time. In some implementations, n2 is less than n1, which can result in a provide a steadier influence on the combined CO waveform by the central arterial continuous cardiac output. However, the derivations can be performed in which n2 is greater than or equal to n1, which can be implemented in certain situations, such as when less computation of the peripheral arterial pressure cardiac output is desired. In some implementations, n2 is between an order of seconds to an order of minutes. In some implementations, n2 is between 1 second and 600 seconds. In various implementations, n2 is about 1 second, n2 is about 5 seconds, n2 is about 10 seconds, n2 is about 15 seconds, n2 is about 20 seconds, n2 is about 25 seconds, n2 is about 30 seconds, n2 is about 35 seconds, n2 is about 40 seconds, n2 is about 45 seconds, n2 is about 50 seconds, n2 is about 55 seconds, n2 is about 60 seconds, n2 is about 75 seconds, n2 is about 90 seconds, n2 is about 105 seconds, n2 is about 120 seconds, n2 is about 135 seconds, n2 is about 150 seconds, n2 is about 165 seconds, n2 is about 180 seconds, n2 is about 195 seconds, n2 is about 210 seconds, n2 is about 225 seconds, n2 is about 240 seconds, n2 is about 255 seconds, n2 is about 270 seconds, n2 is about 285 seconds, n2 is about 300 seconds, n2 is about 315 seconds, n2 is about 330 seconds, n2 is about 345 seconds, n2 is about 360 seconds, n2 is about 375 seconds, n2 is about 390 seconds, n2 is about 405 seconds, n2 is about 420 seconds, n2 is about 435 seconds, n2 is about 450 seconds, n2 is about 465 seconds, n2 is about 480 seconds, n2 is about 495 seconds, n2 is about 510 seconds, n2 is about 525 seconds, n2 is about 540 seconds, n2 is about 555 seconds, n2 is about 570 seconds, n2 is about 585 seconds, or n2 is about 600 seconds.

It is a goal of the current disclosure to derive a combined cardiac output that better mimics the amplitude of a peripherally derived cardiac output but that is centered about the centrally derived cardiac output. It is an additional goal, in some implementations, to reduce the amplitude of the combined cardiac output when the peripherally derived cardiac output is highly fluctuant or when the cardiac index is an aberrant value. The goals are achieved by the determination of the scale and the offset for use in time varying linear scaling as described herein. In some implementations, the scale can be a preset value. In some implementations, the scale is adjusted when the peripherally derived cardiac is highly fluctuant or when the cardiac index is an aberrant value. In some implementations, the offset is determined using the scaling factor, the temporally weighted APCO, and the temporally weighted thermodilution based cardiac output.

The described systems, devices, and methods should not be construed as limiting in any way. Instead, the present disclosure is directed toward all novel and nonobvious features and aspects of the various disclosed systems, devices and methods, alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

Various systems and devices utilized for hemodynamic monitoring can involve performing a procedure within a recipient (e.g., Swan-Ganz catherization). Recipients include (but are not limited to) patients, animal models, or cardiovascular system simulators. Accordingly, in addition to methods of monitoring patients, the systems and devices can be utilized in training or other practice procedures upon animal models or cardiovascular system simulators.

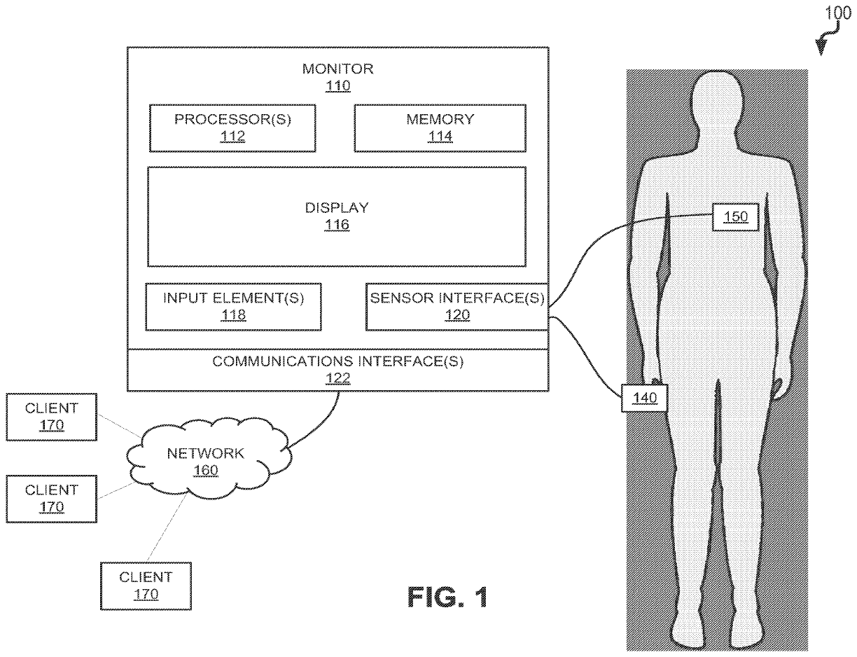

FIG. 1 provides an example of a hemodynamic (HD) monitoring system. The system comprises a HD monitor 110 that can be configured to receive data characterizing various measured physiological parameters of a patient 100 from a first physiological sensor 140 and a second physiological sensor 150. HD monitor 110 can comprise and/or be in communication with a computational processing system 112 (which may have multiple processors, each of which can have multiple processing cores), memory 114 for storing instructions for execution by the computational processing system 112, an electronic visual display 116 for rendering a graphical user interface for displaying information, such as measured parameters or parameters that are derived from measurements obtained from first physiological sensor 140 and/or second physiological sensor 150. Such information can take various forms, including waveforms, numerical indications, categorical indications, and the like. HD monitor 110 can also comprise various interface input elements 118 which can be physically manipulated to affect operation of HD monitor 110 such as which information is being displayed on the display 116 and/or configuration information for first physiological sensor 140 and/or second physiological sensor 150. Display 116 can also comprise a touch-screen interface allowing users to select graphical user interface elements directly.

HD monitor 110 can further include a sensor interface 120 that enables data to be received from and/or transmitted to one or more physiological sensors including first physiological sensor 140 and second physiological sensor 150. Sensor interface 120 can communicate with first physiological sensor 140 and/or second physiological sensor 150 using a physical wired connection and/or using a wireless data protocol (e.g., Bluetooth, WiFi, cellular). HD monitor 110 can also include at least one communications interface 122 that can enable direct or indirect communication with one or more remote client computing systems 170 via a wired and/or wireless network 160. For example, HD monitor 110 can convey and/or exchange data with a remote computing system (e.g., HD monitor 110 can transmit the physiological measurements for storage by a remote database and/or cloud-storage service, the HD monitor 110 can receive contextual and/or historical information about the patient, etc.).

First physiological sensor 140 can be a peripheral artery pressure sensor (e.g., a PAL) or one or more blood pressure cuffs that can be used one an extremity (e.g., digit, toe, arm, leg, etc.). First physiological sensor 140 can be used to derive various hemodynamic parameters, including stroke volume, stroke volume variation, cardiac output, systemic vascular resistance (SVR) and continuous blood pressure (cBP) using an APCO algorithm. The peripheral artery pressure sensor can perform real-time hemodynamic parameter measurements at a sampling rate of, for example, 100 to 1000 times per second.

The second physiological sensor 150 can be a PAC (e.g., Swan-Ganz catheter). Such catheter can be inserted into a pulmonary artery of patient 130 to detect direct, simultaneous measurement of pressures in the right atrium, right ventricle, pulmonary artery, and the filling pressure of the left atrium of patient 130 by way of a thermal filament located on the catheter and using thermodilution principles. The second physiological sensor 150 can also be used to measure CO using bolus thermodilution methods. Second physiological sensor 150 can be used to derive CCO or ICO.

The techniques described herein can be processed by the computational processing system 112 of or in communication with HD monitor 110 and/or such processing many be offloaded to first physiological sensor 140, and/or second physiological sensor 150, and/or a remote client computing device 170 (which may access the underlying data via the communications interface(s) 122).

With the current arrangement, the CCO or ICO algorithm can be performed using data accumulated by second physiological sensor 150 and can be combined with the APCO algorithm as performed using data accumulated by first physiological sensor 140 in order to derive a patient's time varying cardiac output, c(t) having an amplitude akin to the derived APCO and centralized to the derived CCO or ICO. While the following refers to the second physiological sensor 150 as implementing the CCO algorithm, the thermal dilution input measurements may come from a CCO, an ICO, or a mix of ICO or CCO algorithms. In addition, while the following refers to CO algorithm output parameter, the current combination techniques can be applied to other hemodynamic parameter outputs from the algorithms utilizing data input from first physiological sensor 140 and second physiological sensor 150. For example, stroke volume (SV), can be derived from data measurements acquired from first physiological sensor 140 and second physiological sensor 150, which can then be combined utilizing the systems, devices, and methods described herein. CO can also be further utilized to derive cardiac index (CI) and SV. SV can also be further utilized to derive stroke volume index (SVI).

One approach employed by HD monitor 110 can iteratively compute a time-varying linear scaling, A[n], and an offset, B[n], that update an APCO measurement generated by the first physiological sensors 140, where m_{F}[n]=APCOₙ₁, to generate a combined cardiac output \hat{m}[n] as follows

$$\hat{m} [ n ] = A [ n ] {m}_{F} [ n ] + B [ n ] \qquad ( 1 )$$

Note that Equation 1 is a rewritten form of Equation A1.

Parameters A[n] and B[n] can be functions of the past history of CO estimates. These functions can combine with the APCO measurement to yield an enhanced fusedCO parameter. As used herein m_{F} can be referred to as “measurement fast” as consistent with a peripheral artery hemodynamic sensor, and m_{A} can refer to as “measurement accurate” as consistent with a central hemodynamic sensor.

Namely, A[n] and B[n] can be a least mean-square error solution to the following linear equations that arise by replacing m_{A} in eq. 2 with CCO measurements and \hat{m}_{F} with averaged APCO measurements,

$$[ \begin{matrix}{m}_{A} [ 0 ] \\ {m}_{A} [ 1 ] \\ ⋮ \\ {m}_{A} [ n ]\end{matrix} ] = [ \begin{matrix}{\overline{m}}_{F} [ 0 ] & 1 \\ {\overline{m}}_{F} [ 1 ] & 1 \\ ⋮ & \\ {\overline{m}}_{F} [ n ] & 1\end{matrix} ] [ \begin{matrix}A [ n ] \\ B [ n ]\end{matrix} ] \qquad ( 2 )$$

$${m}_{A} [ n ] = [ \begin{matrix}{\overline{m}}_{F} [ n ] & 1\end{matrix} ] [ \begin{matrix}A [ n ] \\ B [ n ]\end{matrix} ]$$

$$where$$

$${{m}_{A} [ n ] = {m}_{A} [ 0 ] , {m}_{A} [ 1 ] , … , {m}_{A} [ n - 1 ] , {m}_{A} [ n ] ]}^{T} \qquad ( 3 )$$

is a sequence of discrete CCO estimates arriving at times,

$${t}_{A} [ 0 ] , {t}_{A} [ 1 ] , … , {t}_{A} [ n - 1 ] , {t}_{A} [ n ] \qquad ( 4 )$$

and where each time value, t_{A}[n], represents the average value of c(t) over an immediately preceding window of time, w_{A}[n], given by the corresponding sequence,

$${w}_{A} [ 0 ] , {w}_{A} [ 1 ] , … , {w}_{A} [ n - 1 ] , {w}_{A} [ n ] \qquad ( 5 )$$

The sequence of APCO estimates,

$${\overline{m}}_{F} [ n ] , {[ {\overline{m}}_{F} [ n ] , {\overline{m}}_{F} [ n - 1 ] , … , {\overline{m}}_{F} [ 1 ] , {\overline{m}}_{F} [ 0 ] ]}^{T} \qquad ( 6 )$$

can represent averages over a time period that is as close as possible to the averaging time window corresponding to m_{A}[n]. An APCO measurement with a line over it, \hat{m}_{F}[k], signifies the averaging time adjustment for m_{F}[k].

An optimum least squares solution for [A[n] B[n]]^{T} can minimize the weighted squared error,

$${min}_{A , B} {ϵ}^{2} = {min}_{A , B} {‖ {m}_{A} - [ \begin{matrix}{\overline{m}}_{F} & 1\end{matrix} ] [ \begin{matrix}A \\ B\end{matrix} ] ‖}_{Ω}^{2} = \qquad ( 7 )$$

$${min}_{A , B} [ {m}_{A}^{T} - [ \begin{matrix}A & B\end{matrix} ] [ \begin{matrix}{\overline{m}}_{F}^{T} \\ {1}^{T}\end{matrix} ] ] Ω [ {m}_{A} - [ \begin{matrix}{\overline{m}}_{F} & 1\end{matrix} ] [ \begin{matrix}A \\ B\end{matrix} ] ]$$

where for clarity, the dependency on n is not shown. The positive definite matrix,

$$Ω = \vert \begin{matrix}ω [ 0 ] & 0 & … & 0 \\ 0 & ω [ 1 ] & ⋱ & ⋮ \\ ⋮ & ⋱ & ⋱ & 0 \\ 0 & … & 0 & ω [ n ]\end{matrix} ⌋ \qquad ( 8 )$$

weights by ω the relative information of the n^{th} measurement. Measurements that are known to be more accurate provide more information and could be weighted higher, while those that are less accurate provide less information and could be weighted lower. For example, if one had the standard deviations for each accurate and averaged fast measurement, σ_{A}[n]+σ_{F}[n], a reasonable choice for each weight could be

$$ω [ n ] = \frac{1}{{σ}_{A} [ n ] + {σ}_{F} [ n ]} \qquad ( 9 )$$

However, if one could classify which measurements were “good” and which were “bad”, another option for the weighting parameters could be

$$ω [ n ] = \{ \begin{matrix}1 & if measurement is good \\ 0 & if measurement is bad\end{matrix} \qquad ( 10 )$$

The important point is that any measure of relative measurement information could be used for the weighting. Weighting can be ignored by setting all weights the same, e.g. ω[n]=1.

For computing the average, HD monitor 110 can, for example, save current and past values of the APCO measurements,

$${m}_{F} [ k ] = {[ {m}_{F} [ k ] , {m}_{F} [ k - 1 ] , … , {m}_{F} [ k - N + 1 ] ]}^{T} \qquad ( 11 )$$

with corresponding sample times,

$${t}_{F} [ k ] = {[ {t}_{F} [ k ] , {t}_{F} [ k - 1 ] , {t}_{F} [ k - 2 ] , … , {t}_{F} [ k - N + 1 ] ]}^{T} \qquad ( 12 )$$

and with corresponding averaging window lengths, t_{F}[k],

$${w}_{F} [ k ] = {[ {w}_{F} [ k ] , {w}_{F} [ k - 1 ] , {w}_{F} [ k - 2 ] , … , {w}_{F} [ k - N + 1 ] ]}^{T} \qquad ( 13 )$$

Measurement m_{F}[k] corresponds to time t_{F}[k]−_{F}\ddot{ω}]/2. N is sufficiently large to cover the worst case delay between a CCO measurement and an APCO measurement ω, plus the worst case CCO averaging time window, [n]. Generally, the averaging window lengths for an APCO algorithm are constant.

The discrete time index, k, is a frequency for APCO estimates and is different from index n, which is a frequency for CCO estimates. In general, APCO and CCO algorithm estimates are (a) not synchronized to a common sampling clock, (b) do not have the same sample time intervals, and (c) nor do they have simple integer or rational fraction related sample time intervals.

Multiplying both sides of eq. 2 by Ω and transposing the right-hand side matrix results in the following equivalent linear equations,

$$[ \begin{matrix}{S}_{FA} [ n ] \\ {S}_{A} [ n ]\end{matrix} ] = [ \begin{matrix}{S}_{FF} [ n ] & {S}_{F} [ n ] \\ {S}_{F} [ n ] & M [ n ]\end{matrix} ] [ \begin{matrix}A [ n ] \\ B [ n ]\end{matrix} ] \qquad ( 14 )$$

$$where$$

$${S}_{F} [ n ] = {\sum }_{k = 0}^{n} {ω}^{2} [ k ] {\overline{m}}_{F} [ k ] \qquad ( 15 )$$

$${S}_{FF} [ n ] = {\sum }_{k = 0}^{n} {ω}^{2} [ k ] {\overline{m}}_{F}^{2} [ k ] \qquad ( 16 )$$

$${S}_{FA} [ n ] = {\sum }_{k = 0}^{n} {ω}^{2} [ k ] {\overline{m}}_{F} [ k ] {m}_{A} [ k ] \qquad ( 17 )$$

$${S}_{A} [ n ] = {\sum }_{k = 0}^{n} {ω}^{2} [ k ] {m}_{A} [ k ] \qquad ( 18 )$$

$$M [ n ] = {\sum }_{k = 0}^{n} {ω}^{2} [ k ] \qquad ( 19 )$$

All key parameters used to solve for (adapt) A[n] and B[n] can be updated iteratively for new CCO, m_{A}, and averaged APCO, \hat{m}_{F}, measurements as follows, and discounting past measurements using a “forgetting parameter”, 0<γ<1,

$${S}_{F} [ n ] = γ {S}_{F} [ n - 1 ] + ( 1 - γ ) \times {M}_{F} [ n ] \qquad ( 20 )$$

$${S}_{FF} [ n ] = γ {S}_{FF} [ n - 1 ] + ( 1 - γ ) \times {M}_{F} [ n ] \times {M}_{F} [ n ] \qquad ( 21 )$$

$${S}_{FA} [ n ] = γ {S}_{FA} [ n - 1 ] + ( 1 - γ ) \times {M}_{F} [ n ] \times {M}_{A} [ n ] \qquad ( 22 )$$

$${S}_{A} [ n ] = γ {S}_{A} [ n - 1 ] + ( 1 - γ ) \times {M}_{A} [ n ] \qquad ( 23 )$$

$$M [ n ] = γ M [ n - 1 ] + ( 1 - γ ) \qquad ( 24 )$$

Setting γ to a small value or “high forgetting” places more weight on the most recent measurement relative to the past, while setting it to a value near 1 or “low forgetting” places more weight on the past.

Referring back to eq. A1 and eq. 1, a goal of the disclosure is to derive a combined cardiac output that better mimics the amplitude of a peripherally derived cardiac output but that is centered about the centrally derived cardiac output. To achieve the amplitude, in some implementations, scaling factor A is set to a preset value. Having a preset value ensures the amplitude of FusedCOₙ₁ follows the amplitude and responsiveness of the computed APCOₙ₁. In theory, any preset value can be utilized and can be preset to a desired visualization of the amplitude of FusedCOₙ₁. For instance, if the scaling factor is preset to a value of 1, the amplitude will be the same as APCOₙ₁. If the scaling factor is preset to a value of 2, the amplitude will be double of that as APCOₙ₁. And likewise, if the scaling factor is preset to a value of 0.5, the amplitude will be half of that as APCOₙ₁.

In some implementations, scaling factor A is set to a preset value of any number between 0.1 and 10. In various implementations, scaling factor A is set to a preset value at or about 0.1, scaling factor A is set to a preset value at or about 0.2, scaling factor A is set to a preset value at or about 0.3, scaling factor A is set to a preset value at or about 0.4, scaling factor A is set to a preset value at or about 0.5, scaling factor A is set to a preset value at or about 0.6, scaling factor A is set to a preset value at or about 0.7, scaling factor A is set to a preset value at or about 0.8, scaling factor A is set to a preset value at or about 0.9, scaling factor A is set to a preset value at or about 1.0, scaling factor A is set to a preset value at or about 1.1, scaling factor A is set to a preset value at or about 1.2, scaling factor A is set to a preset value at or about 1.3, scaling factor A is set to a preset value at or about 1.4, scaling factor A is set to a preset value at or about 1.5, scaling factor A is set to a preset value at or about 1.6, scaling factor A is set to a preset value at or about 1.7, scaling factor A is set to a preset value at or about 1.8, scaling factor A is set to a preset value at or about 1.9, scaling factor A is set to a preset value at or about 2.0, scaling factor A is set to a preset value at or about 3.0, scaling factor A is set to a preset value at or about 4.0, scaling factor A is set to a preset value at or about 5.0, scaling factor A is set to a preset value at or about 6.0, scaling factor A is set to a preset value at or about 7.0, scaling factor A is set to a preset value at or about 8.0, scaling factor A is set to a preset value at or about 9.0, or scaling factor A is set to a preset value at or about 10.0.

Referring back to eq. A1 and eq. 1, to achieve an offset that is centered about the centrally derived cardiac output, offset factor B can be computed using the weighted past measurements of APCO and CCO. In some implementations, offset factor B is computed as follows:

$$B = \frac{( {S}_{FA} - A \times {S}_{FF} - {S}_{A} + A \times {S}_{F} )}{( {S}_{F} - M - β )} \qquad ( 25 )$$

where β is an equation regularization parameter, which is an empirical value that is used as described in eq. 26. In some implementations, β=0.

In some situations, it is desired to alter the preset scaling value A to yield a more smoothened combined CO. For various reasons, the amplitude of APCOₙ₁ can be highly fluctuant. Thus, in some implementations, the waveform of combined CO values is smoothened by reducing the scaling factor. Any mechanism to reduce the scaling factor can be utilized, including (but not limited to) reducing the scaling factor by a numerical factor and reducing the scaling factor based on a ratio of CCO to APCO (which can be weighted by prior measurements).

Other linear equation solution methods that can be utilized by HD monitor 110 include, without limitation, Gaussian elimination, QR factorization, Householder reflectors, and the like. Solution via multiplication by orthogonal matrices (“Householder reflectors”), for example, is a numerically stable method that does not amplify errors.

Adding a “regularization” term to the weighted least squares optimization problem of eq. 7 provides a means to encourage offset factor B to stay near to 0:

$${min}_{A , B} {ϵ}^{2} = {min}_{A , B} \{ {‖ {\overline{m}}_{A} - [ \begin{matrix}{\overline{m}}_{F} & 1\end{matrix} ] [ \begin{matrix}A \\ B\end{matrix} ] ‖}^{2} + {β ( B )}^{2} \} \qquad ( 26 )$$

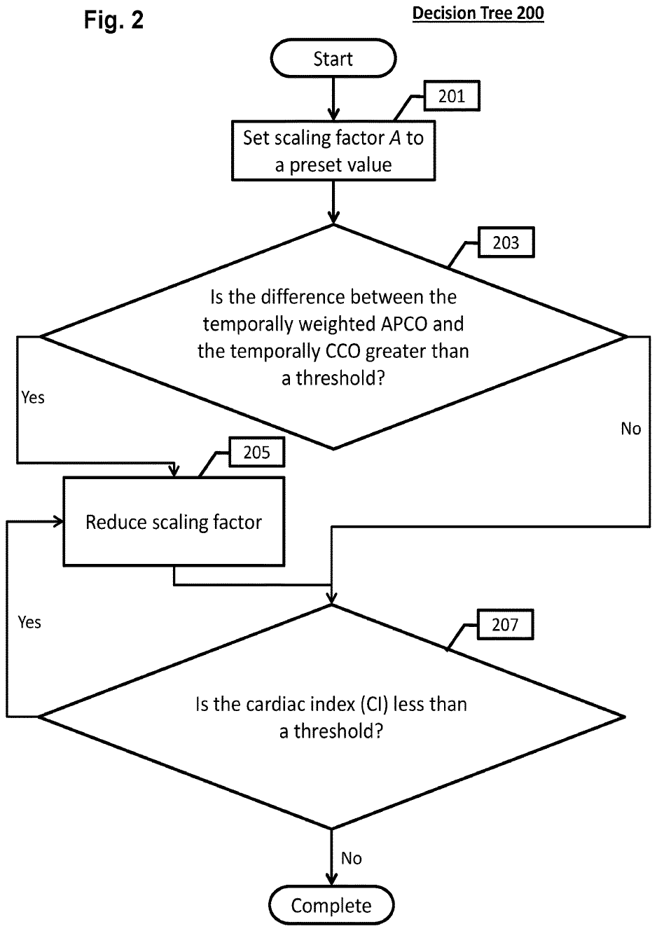

FIG. 2 provides an example of decision tree flow chart for dynamic scaling to yield a more a smoothened signal of FusedCOₙ₁ by altering a preset scaling value. Decision Tree 200 can be used in some implementations of computing a combined CO. At step 201, scaling factor A is set to a preset value. As discussed herein, the preset value can be any value to yield a desired visualization of the amplitude of FusedCOₙ₁.

The derivation of APCO as compared to the steadier and more consistent CCO value is assessed. Accordingly, at decision step 203, the difference between the temporally weighted APCO and the temporally CCO greater is determined, as follows:

$${CO}_{diff} = {S}_{F} - {S}_{A} \qquad ( 27 )$$

If the difference is greater than a threshold, indicating that the APCO is highly fluctuant, the then preset scaling factor is reduced at step 205. Any reduction of the preset scaling factor can be utilized, including (but not limited to) reducing the preset scaling factor by a numerical factor or by a ratio of CCO measurements to APCO measurements. In some situations, multiple thresholds can be utilized, such that when CO_{diff} is greater than the higher threshold, the preset factor is more greatly reduced (or vice versa, when CO_{diff} is less than the higher threshold but still higher than a lower threshold, the preset factor is less greatly reduced). In some implementations, when CO_{diff} is greater than or equal to a threshold, the scaling factor is reduced by multiplying the preset scaling factor with S_{A}/S_{F}, as follows:

$${A}_{reduced} = {A}_{preset} \times \frac{{s}_{A}}{{s}_{F}} \qquad ( 28 )$$

In some implementations, when the difference between the temporally weighted APCO (S_{F}) and the temporally weighted thermodilution based cardiac output (S_{A}) is greater than or equal to a lower threshold but less than a higher threshold, the scaling factor is reduced as follows:

$${A}_{reduced} = {A}_{preset} [ \frac{{S}_{A}}{{S}_{F}} \times ( {CO}_{diff} - {CO}_{lowThresh} ) + ( {CO}_{highThresh} - {CO}_{diff} ) ] \qquad ( 29 )$$

where CO_{highThresh} is the higher threshold, CO_{lowThresh} is the lower threshold. In some implementations, just a single threshold is used along with eq. 28. In some implementations, two threshold are used, in which eq. 28 is used when CO_{diff} is greater than CO_{highThresh} and eq. 29 is utilized when CO_{diff} is greater than or equal to CO_{lowThresh} but less than CO_{highThresh}.

If CO_{diff} is not greater than threshold or after reducing the preset scaling factor when CO_{diff} was greater than a threshold, then the cardiac index (CI) is assessed to determine whether the it is an aberrant value. CI can be determined as follows:

$${FusedCI}_{n 1} = \frac{{FusedCO}_{n 1}}{BSA} \qquad ( 30 )$$

where BSA is the body surface area of a patient. If the preset scaling factor has not been reduced, then FusedCO is computed using the preset scaling factor. If the preset scaling factor has already been reduced, then FusedCO is computed using the reduced scaling factor.

Generally, CI for a healthy individual is between 2.5 and 4.2 L/min/m². A lower value (e.g., under 2.0 L/min/m²) might mean the patient is undergoing cardiogenic shock. However, in practice, a low value for FusedCIₙ₁ can be aberrant and arise due to a transient low fluctuation of APCOₙ₁. Accordingly, at decision step 207, FusedCIₙ₁ is derived to determine whether the CI value is below a threshold indicating the value is aberrant. If the CI value is not below a threshold, the decision factor is complete and no further reduction of scaling factor A is needed. If the CI value is below a threshold, then at step 205, the scaling factor is reduced. Any reduction of the preset scaling factor can be utilized. In some implementations, scaling factor A is reduced by a numerical factor. The numerical factor used to reduce scaling factor A can be any number, depending on the amount of reduction of scale that is desired. In some implementations, the numerical factor used to reduce scaling factor A is greater than 1 and less than or equal to 2, yielding a small reduction. In some implementations, the numerical factor used to reduce scaling factor A is greater than 2, yielding a greater reduction. In some implementations, multiple thresholds can be utilized, in which the lower the threshold, the numerical value factor used to reduce scaling factor A is greater.

After completing step 205 to reduce scaling factor A, decision step 207 can be performed again to assess whether the cardiac index (CI) is still below a threshold. If, upon reassessment it is determined that the cardiac index (CI) is still below a threshold, step 205 and 207 can be repeated. In some implementations, step 207 and 205 can be iteratively repeated until CI is greater than the threshold. In some implementations, step 207 and 205 can be performed for a preset number of iterations. The preset number of iterations can be any whole number value. In some implementations, the preset number of iterations is between 1 and 10. In various implementations, the preset number of iterations is 1, the preset number of iterations is 2, the preset number of iterations is 3, the preset number of iterations is 4, the preset number of iterations is 5, the preset number of iterations is 6, the preset number of iterations is 7, the preset number of iterations is 8, the preset number of iterations is 9, or the preset number of iterations is 10. Upon completion of Decision Tree 200, eq. A1 (or eq. 1) can be performed to yield a combined CO value.

HD monitor 110 can implement the techniques provided herein using a variety of software and/or hardware implementations. In one implementation utilizing software, a module can include a measurement “Combiner class” that implements the adaptive least squares mathematics to combine a first algorithm parameter (e.g., APCO) with a second algorithm parameter (e.g., CCO). The implementation provides an option to use a forgetting factor γ, or to use a finite number of past accurate algorithm estimates. In the former case, the number of past measurements is a function of γ.

The Combiner class can assume that the first algorithm parameter and the second algorithm parameter may arrive asynchronously at different times. There can be separate interface functions to update the algorithm with these measurements. A first function (e.g., function UpdateFirst(c, t, w)) can provide the means to inform the algorithm of a new first algorithm parameter m along with the measurement time, t, and the averaging time window, w. The measurement represents time window [t−w,t]. A second function (e.g., UpdateSecond (c, t, w)) is an analogous function to inform the algorithm of a new second algorithm parameter. A third function (e.g., function Combine( )) can return a combined measurement.

Methods of monitoring can be performed utilizing the systems and devices. Generally, a patient can be monitored utilizing a set of sensors for gathering data, a computational system for processing data to yield a combined output parameter, and a system or device to visualize and/or comprehend the combined output parameter.

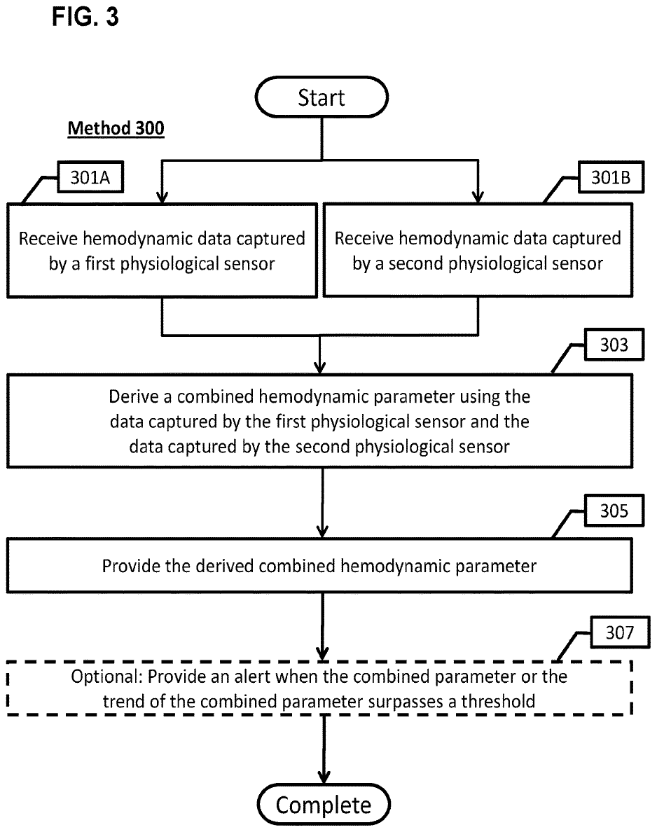

Provided in FIG. 3 is a method for monitoring a patient that utilizes two sources of data to derive a combined parameter, which can be implemented on a computational device. Method 300 can begin by receiving (301A) hemodynamic data captured by a first physiological sensor and receiving (301B) hemodynamic data captured by a second physiological sensor. Generally, any two or more sensors that can yield the same hemodynamic parameter can be utilized. In some implementations, the first sensor is a peripheral hemodynamic sensor and the second sensor is a central hemodynamic sensor, or vice versa. In some implementations, the first sensor is an extracorporeal hemodynamic sensor and the second sensor is an internal hemodynamic sensor, or vice versa. In some implementations, the first sensor is a faster acquisition sensor and the second sensor is a more accurate acquisition sensor, or vice versa. In some implementations, the first sensor is a PAL or a blood pressure cuff and the second sensor is a pulmonary artery catheter, or vice versa.

The first sensor and the second sensor can capture data to yield any hemodynamic parameter for combining. Hemodynamic parameters that can be combined include (but are not limited to) blood pressure, mean arterial pressure (MAP), systolic pressure variation (SPV), pulse pressure variation (PPV), cardiac output (CO), cardiac index (CI), stroke volume (SV), stroke volume variation (SVV), stroke volume index (SVI), systemic vascular resistance (SVR), systemic vascular resistance index (SVRI), pulmonary vascular resistance (PVR), and pulmonary vascular resistance index (PVRI).

Upon receiving the hemodynamic data from the first physiological sensor and from the second physiological sensor, Method 300 derives (303) a combined parameter using the captured data. Any means for combining a hemodynamic parameters from two data sources can be utilized. In some implementations, the combined parameter is derived using least squares fitting and a time-varying linear scaling equation. Method 300 further provides (305) the combined hemodynamic parameter. Any means for providing the combined hemodynamic parameter can be utilized. Examples of providing the combined hemodynamic parameter include (but are not limited to) displaying the parameter on an electronic display, printing out the parameter, loading the parameter onto a memory, storing the parameter in physical data persistence, transmitting the parameter to a remote computing device, and utilizing the parameter in a downstream computation. Optionally, an alert or fault can be provided (307) when the combined parameter or the trend of the combined parameter surpasses a threshold. For instance, if the CO or the CI falls below a low threshold, an alert or a fault can be provided, which may be an alert or a fault to indicate cardiogenic shock. An alert can also be provided if the CO or the CI rises above a high threshold Likewise, an alert can be provided if a CO or a CI declines at a rate greater than a declination threshold. And an alert can be provided if a CO or a CI ascends at a rate greater than an ascension threshold.

Systems and methods can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, can include machine instructions for a programmable processor, and/or can be implemented in a high-level procedural language, an object-oriented programming language, a functional programming language, a logical programming language, and/or in assembly/machine language. As used herein, the term “computer-readable medium” refers to any computer program product, apparatus and/or device, such as for example magnetic discs, optical disks, solid-state storage devices, memory, and Programmable Logic Devices (PLDs), used to provide machine instructions and/or data to a programmable data processor, including a machine-readable medium that receives machine instructions as a computer-readable signal. The term “computer-readable signal” refers to any signal used to provide machine instructions and/or data to a programmable data processor. The computer-readable medium can store such machine instructions non-transitorily, such as for example as would a non-transient solid-state memory or a magnetic hard drive or any equivalent storage medium. The computer-readable medium can alternatively or additionally store such machine instructions in a transient manner, such as for example as would a processor cache or other random-access memory associated with one or more physical processor cores.

The computer components, software modules, functions, data stores and data structures described herein can be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality can be located on a single computer or distributed across multiple computers depending upon the situation at hand.

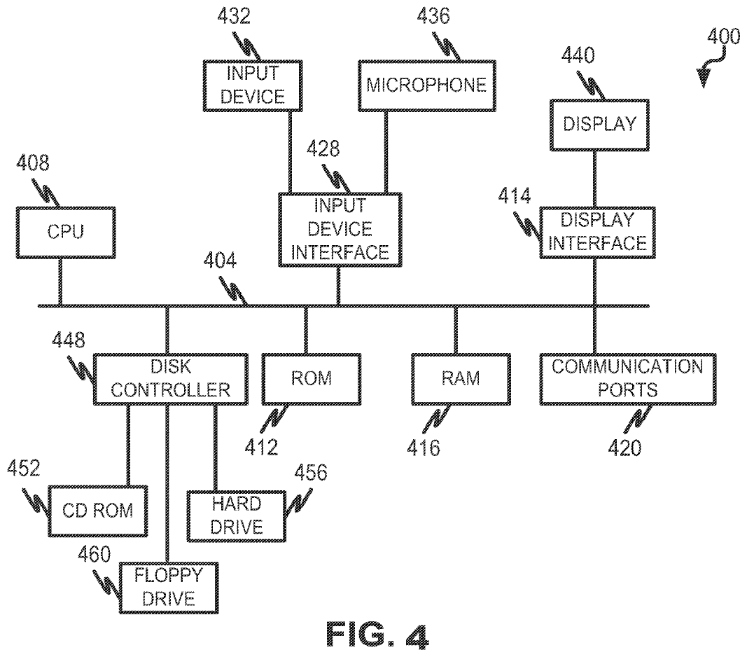

FIG. 4 is a diagram 400 illustrating an example of a computing device architecture for implementing various aspects described herein. A bus 404 can serve as the information highway interconnecting the other illustrated components of the hardware. A processing system 408 labeled CPU (central processing unit) (e.g., one or more computer processors/data processors at a given computer or at multiple computers), can perform calculations and logic operations required to execute a program. A non-transitory processor-readable storage medium, such as read only memory (ROM) 412 and random-access memory (RAM) 416, can be in communication with the processing system 408 and can include one or more programming instructions for the operations specified here. Optionally, program instructions can be stored on a non-transitory computer-readable storage medium such as a magnetic disk, optical disk, recordable memory device, flash memory, solid-state or other physical storage medium.

In one example, a disk controller 448 can interface one or more optional disk drives to the system bus 504. These disk drives can be external or internal floppy disk drives such as 460, external or internal CD-ROM, CD-R, CD-RW or DVD, or solid-state drives such as 452, or external or internal hard drives 456. As indicated previously, these various disk drives 452, 456, 460 and disk controllers are optional devices. The system bus 404 can also include at least one communication port 420 to allow for communication with external devices either physically connected to the computing system or available externally through a wired or wireless network. In some cases, the communication port 420 includes or otherwise comprises a network interface.

To provide for interaction with a user, the subject matter described herein can be implemented on a computing device having a display device 440 (e.g., a CRT (cathode ray tube) or LCD (liquid crystal display) monitor, touchscreen, etc.) for displaying information obtained from the bus 404 to the user and an input device 432 such as keyboard and/or a pointing device (e.g., a mouse or a trackball) and/or a touchscreen by which the user can provide input to the computer. Other kinds of input devices 432 can be used to provide for interaction with a user as well; for example, feedback provided to the user can be any form of sensory feedback (e.g., visual feedback, auditory feedback by way of a microphone 436, or tactile feedback); and input from the user can be received in any form, including acoustic, speech, or tactile input. In the input device 432 and the microphone 436 can be coupled to and convey information via the bus 404 by way of an input device interface 428. Other computing devices, such as dedicated servers, can omit one or more of the display 440 and display interface 424, the input device 432, the microphone 436, and input device interface 428.

In the descriptions above and in the claims, phrases such as “at least one of” or “one or more of” can occur followed by a conjunctive list of elements or features. The term “and/or” can also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

### Data Examples of Parameter Derivation

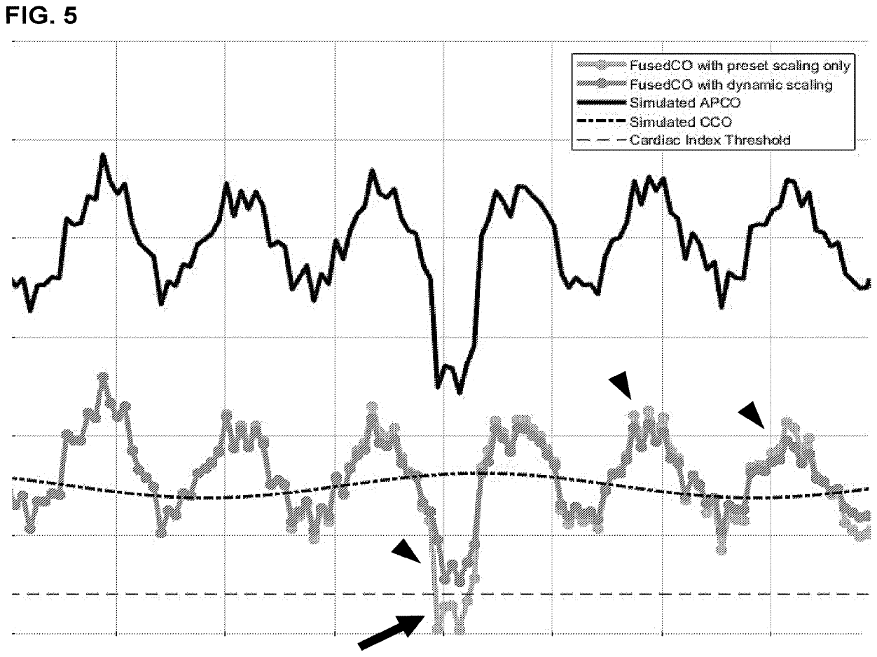

FIG. 5 provides an example of combining cardiac output parameters derived from two physiological sensors. The example of FIG. 5 also compares the use of preset scaling only and dynamic scaling using a preset scaling factor that is reduced upon hemodynamic parameters surpassing a threshold.

In this example, simulated data is utilized to derive an APCO and a CCO. The derived APCO parameter is derived from data simulated to be captured by a PAL and the derived CCO parameter is derived from data simulated to be captured by a PAC. As readily apparent in the data, the APCO waveform is highly dynamic with larger amplitudes and highly responsive due to high frequency of derivation. On the other hand, the CCO waveform is much more muted with smaller amplitudes and less responsive due to lower frequency derivation. The CCO waveform, however, captures a more centralized CO and thus is more accurate.

As stated herein, it is a goal of the current disclosure to derive a combined CO that better mimics the amplitude and responsiveness of a peripherally derived cardiac output but that is centered about the centrally derived cardiac output. As the simulated data shows in FIG. 5, the FusedCO with preset scaling only is a combined CO that mimics the amplitude and responsiveness of the APCO waveform and is centralized to the CCO waveform.

Another goal of the current disclosure, in some implementations, is to reduce the amplitude of the combined cardiac output when the peripherally derived cardiac output is highly fluctuant or when the cardiac index is an aberrant value. As the simulated data shows in FIG. 5, the FusedCO with dynamic scaling achieves this goal. This combined CO parameter is derived utilizing a preset scaling factor that is reduced when the difference APCO between CCO is greater than a threshold. At points along the FusedCO with dynamic scaling waveform, as indicated by the arrow heads, the amplitude is reduced when the difference APCO between CCO is greater than a threshold, resulting in a smoothened waveform. Further, at one point within the FusedCO with dynamic scaling waveform, as indicated by the arrow, the amplitude is reduced when the computed CI is below the cardiac index threshold, resulting in a more accurate CO waveform. It is noted that FusedCI is not depicted, but the cardiac index threshold shown was converted back into a CO threshold utilizing a simulated patient body surface area.

### EXAMPLES

Example 1. A method of hemodynamic monitoring of a patient to provide a combined cardiac output measurement from two hemodynamic sensors, the method comprising: sensing, using a peripheral arterial pressure sensor, a peripheral arterial pressure signal of a patient; deriving, using a computational processing system and an arterial pressure-based cardiac output (APCO) algorithm, an APCO of the patient based upon the peripheral arterial pressure signal; sensing, using a pulmonary artery catheter, a central blood flow signal of the patient, deriving, using the computational processing system and a thermodilution based cardiac output algorithm, a thermodilution based cardiac output of the patient based upon the central blood flow signal;
deriving, using the computational processing system, a time-varying linear scaling equation,
wherein the derivation of the time-varying linear scaling equation comprises:
   determining a scaling factor and an offset factor;
   wherein determining the scaling factor comprises determining whether the scaling factor is a preset value or a computed value using a temporally weighted APCO and a temporally weighted thermodilution based cardiac output; and
   wherein determining the offset factor comprises using the scaling factor, the temporally weighted APCO, and the temporally weighted thermodilution based cardiac output; and
computing, using the computational processing system and the derived time-varying linear scaling equation, a combined cardiac output, wherein computing the combined cardiac output comprises applying the scaling factor and the offset factor to the APCO.

Example 2. The method of claim 1, wherein the APCO is iteratively derived at a first frequency and the thermodilution based cardiac output is iteratively derived at a second frequency.

Example 3. The method of claim 2, wherein the combined cardiac output is iteratively computed at the first frequency.

Example 4. The method of claim 2 or 3, wherein the scaling factor and the offset factor are each iteratively determined at the second frequency.

Example 5. The method of claim 2, 3, or 4, wherein the time-varying linear scaling equation is:

$${FusedCO}_{n 1} = {A}_{n 2} \times {APCO}_{n 1} + {B}_{n 2}$$

wherein n1 is the first frequency, n2 is the second frequency, A is the scaling factor, B is the offset factor, and FusedCO is the combined cardiac output.

Example 6. The method of any one of claims 2 to 5, wherein the first frequency is greater than the second frequency.

Example 7. The method of any one of claims 1 to 6, wherein the scaling factor is determined to be the preset value when the difference between the temporally weighted APCO and the temporally weighted thermodilution based cardiac output is less than a first cardiac output threshold and a first combined cardiac index of the patient is greater than a cardiac index threshold. Example 8. The method of claim 7, wherein the preset value is 1.

Example 9. The method of claim 7 or 8, wherein the scaling factor is determined to be the computed value when the difference between the temporally weighted APCO and the temporally weighted thermodilution based cardiac output is greater than the first cardiac output threshold.

Example 10. The method of claim 9, wherein when the difference between the temporally weighted APCO and the temporally weighted thermodilution based cardiac output is greater than the first cardiac output threshold and less than a second cardiac output threshold, the computed value of the scaling factor is computed as follows:

$${A}_{reduced} = {A}_{preset} [ \frac{{S}_{A}}{{S}_{F}} \times ( {CO}_{diff} - {CO}_{lowThresh} ) + ( {CO}_{highThresh} - {CO}_{diff} ) ]$$

wherein A_{reduced} is the computed value of the scaling factor; Aₚᵣₑₛₑₜ is the preset value of the scaling factor; S_{F} is the temporally weighted APCO; S_{A} is the temporally weighted thermodilution based cardiac output; COthresh1 is the first cardiac output threshold; COthresh2 is the second cardiac output threshold; COthresh1<COthresh2; and

CO_{diff}=S_{F}−S_{A}.

Example 11. The method of claim 9, wherein when the difference between the temporally weighted APCO and the temporally weighted thermodilution based cardiac output (S_{A}) is greater than or equal to the second cardiac output threshold, the computed value of the scaling factor is computed as follows:

$${A}_{reduced} = {A}_{preset} \times \frac{{S}_{A}}{{S}_{F}}$$

wherein A_{reduced} is the computed value of the scaling factor; Aₚᵣₑₛₑₜ is the preset value of the scaling factor; S_{F} is the temporally weighted APCO and S_{A} is the temporally weighted thermodilution based cardiac output.

Example 12. The method of claim 10 or 11, wherein the computed value of the scaling factor is further reduced by a first numerical value when an updated combined cardiac index is less than the cardiac index threshold; wherein the updated combined cardiac index is computed using the computed value of the scaling factor of claim 10 or claim 11.

Example 13. The method of claim 12, wherein the computed value of the scaling factor is further reduced by a second numerical value when a further updated combined cardiac index is less than the cardiac index threshold; wherein the further updated combined cardiac index is computed using the computed value of the scaling factor of claim 12.

Example 14. The method of claim 7 or 8, wherein the scaling factor is determined to be the computed value when the first combined cardiac index of the patient is less than the cardiac index threshold; and wherein when the first combined cardiac index of the patient is less than the cardiac index threshold, the computed value of the scaling factor is computed by reducing the preset scaling factor by a first numerical factor.

Example 15. The method of 14, wherein the computed value of the scaling factor is further reduced by a second numerical value when an updated combined cardiac index is less than the cardiac index threshold; wherein the further updated combined cardiac index is computed using the computed value of the scaling factor of claim 14.

Example 16. The method of any one of claims 12 to 15, wherein the combined cardiac index or the updated combined cardiac index is computed as follows:

$${FusedCI}_{n 1} = \frac{{FusedCO}_{n 1}}{BSA}$$

wherein BSA is a body surface area of the patient and FusedCI is the combined cardiac index or the updated combined cardiac index.

Example 17. The method of any one of claims 1 to 16, wherein the offset is determined by the following equation:

$$B = \frac{( {S}_{FA} - A \times {S}_{FF} - {S}_{A} + A \times {S}_{F} )}{( {S}_{F} - M - β )}$$

wherein B is the offset, A is the scaling, β is a regularization parameter, where S_{F} is the temporally weighted APCO, S_{A} is the temporally weighted CCO, S_{FA} is the temporally weighted inner product of APCO and CCO, S_{FF} is the temporally weighted inner product of APCO with itself, and M is the temporally weighted counter.

Example 18. The method of claim 17, wherein β≥0.

Example 19. The method of claim 17, wherein S_{F}, S_{FF}, S_{FA}, S_{A}, and M, are computed for each thermodilution based cardiac output measurement as follows:

$${S}_{F} [ n ] = γ {S}_{F} [ n - 1 ] + ( 1 - γ ) \times {M}_{F} [ n ] ;$$

$${S}_{FF} [ n ] = γ {S}_{FF} [ n - 1 ] + ( 1 - γ ) \times {M}_{F} [ n ] \times {M}_{F} [ n ] ;$$

$${S}_{FA} [ n ] = γ {S}_{FA} [ n - 1 ] + ( 1 - γ ) \times {M}_{F} [ n ] \times {M}_{A} [ n ] ;$$

$${S}_{A} [ n ] = γ {S}_{A} [ n - 1 ] + ( 1 - γ ) \times {M}_{A} [ n ] ;$$

$$M [ n ] = γ M [ n - 1 ] + ( 1 - γ ) .$$

Example 20. The method of claim 19, wherein initial values are as follows: S_{F}=0, S_{FF}=0, S_{A}=0, S_{FA}=0, M=0, and γ is a forgetting factor that is >0 and <1.

Example 21. The method of any one of claims 1 to 20 further comprising:

displaying the combined cardiac output on an electronic visual display.

Example 22. The method of any one of claims 1 to 21 further comprising:

providing an alert or fault if the combined cardiac output or a computed combined cardiac index of the patient is above a high threshold or below a low threshold.

Example 23. The method of any one of claims 1 to 22 further comprising:

providing an alert or fault if the combined cardiac output or a computed combined cardiac index declines at a rate greater than a declination threshold or ascends at a rate greater than an ascension threshold.

Example 24. The method of any one of claims 1 to 23, wherein the peripheral arterial pressure sensor is a peripheral arterial line catheter.

Example 25. The method of any one of claims 1 to 23, wherein the peripheral arterial pressure sensor is a blood pressure cuff.

Example 26. A system for hemodynamic monitoring of a patient to provide a combined cardiac output measurement from two hemodynamic sensors, the system comprising:

a peripheral arterial pressure sensor for sensing a peripheral arterial pressure signal;
a pulmonary artery catheter for sensing a central blood flow signal; and
a computational processing system in operable connection with the peripheral arterial pressure sensor and the pulmonary artery catheter; the computational processing system comprising:
a processor system; and
a memory system comprising one or more applications that can direct the processor system to:
   derive, using an arterial pressure-based cardiac output (APCO) algorithm, an APCO based upon the peripheral arterial pressure signal;
   derive, using a thermodilution based cardiac output algorithm, a thermodilution based cardiac output based upon the central blood flow signal;
   derive a time-varying linear scaling equation, wherein the derivation of the time-varying linear scaling equation comprises:
      determining a scaling factor and an offset factor, wherein determining the scaling factor comprises determining whether the scaling factor is a preset value or a computed value using a temporally weighted APCO and a temporally weighted thermodilution based cardiac output, and wherein determining the offset factor comprises using the scaling factor, the temporally weighted APCO, and the temporally weighted thermodilution based cardiac output; and
   compute, using the derived time-varying linear scaling equation, a combined cardiac output, wherein computing the combined cardiac output comprises applying a scaling factor and an offset factor to the APCO.

Example 27. The system of claim 26, wherein the APCO is iteratively derived at a first frequency and the thermodilution based cardiac output is iteratively derived at a second frequency.

Example 28. The system of claim 27, wherein the combined cardiac output is iteratively computed at the first frequency.

Example 29. The system of claim 27 or 28, wherein the scaling factor and the offset factor are each iteratively determined at the second frequency.

Example 30. The system of claim 27, 28, or 29, wherein the time-varying linear scaling equation is:

$${FusedCO}_{n 1} = {A}_{n 2} \times {APCO}_{n 1} + {B}_{n 2}$$

wherein n1 is the first frequency, n2 is the second frequency, A is the scaling factor, B is the offset factor, and FusedCO is the combined cardiac output.

Example 31. The system of any one of claims 27 to 30, wherein the first frequency is greater than the second frequency.

Example 32. The system of any one of claims 26 to 31, wherein the scaling factor is determined to be the preset value when the difference between the temporally weighted APCO and the temporally weighted thermodilution based cardiac output is less than a first cardiac output threshold and a first combined cardiac index of the patient is greater than a cardiac index threshold.

Example 33. The system of any one of claim 32, wherein the preset value is 1.

Example 34. The system of claim 32 or 33, wherein the scaling factor is determined to be the computed value when the difference between the temporally weighted APCO and the temporally weighted thermodilution based cardiac output is greater than the first cardiac output threshold.

Example 35. The system of claim 34, wherein when the difference between the temporally weighted APCO and the temporally weighted thermodilution based cardiac output is greater than the first cardiac output threshold and less than a second cardiac output threshold, the computed value of the scaling factor is computed as follows:

$${A}_{reduced} = {A}_{preset} [ \frac{{S}_{A}}{{S}_{F}} \times ( {CO}_{diff} - {CO}_{lowThresh} ) + ( {CO}_{highThresh} - {CO}_{diff} ) ]$$

wherein A_{reduced} is the computed value of the scaling factor; Aₚᵣₑₛₑₜ is the preset value of the scaling factor; S_{F} is the temporally weighted APCO; S_{A} is the temporally weighted thermodilution based cardiac output; COthresh1 is the first cardiac output threshold; COthresh2 is the second cardiac output threshold; COthresh1<COthresh2; and

CO_{diff}=S_{F}−S_{A}.

Example 36. The system of claim 34, wherein when the difference between the temporally weighted APCO and the temporally weighted thermodilution based cardiac output (S_{A}) is greater than or equal to the second cardiac output threshold, the computed value of the scaling factor is computed as follows:

$${A}_{reduced} = {A}_{preset} \times \frac{{S}_{A}}{{S}_{F}}$$

wherein A_{reduced} is the computed value of the scaling factor; Aₚᵣₑₛₑₜ is the preset value of the scaling factor; S_{F} is the temporally weighted APCO and S_{A} is the temporally weighted thermodilution based cardiac output.

Example 37. The system of claim 35 or 36, wherein the computed value of the scaling factor is further reduced by a first numerical value when an updated combined cardiac index is less than the cardiac index threshold; wherein the updated combined cardiac index is computed using the computed value of the scaling factor of claim 36 or claim 36.

Example 38. The system of claim 37, wherein the computed value of the scaling factor is further reduced by a second numerical value when a further updated combined cardiac index is less than the cardiac index threshold; wherein the further updated combined cardiac index is computed using the computed value of the scaling factor of claim 37.

Example 39. The system of claim 32 or 33, wherein the scaling factor is determined to be the computed value when the first combined cardiac index of the patient is less than the cardiac index threshold; and wherein when the first combined cardiac index of the patient is less than the cardiac index threshold, the computed value of the scaling factor is computed by reducing the preset scaling factor by a first numerical factor.

Example 40. The system of 39, wherein the computed value of the scaling factor is further reduced by a second numerical value when an updated combined cardiac index is less than the cardiac index threshold; wherein the further updated combined cardiac index is computed using the computed value of the scaling factor of claim 39.

Example 41. The system of any one of claims 37 to 40, wherein the combined cardiac index or the updated combined cardiac index is computed as follows:

$${FusedCI}_{n 1} = \frac{{FusedCO}_{n 1}}{BSA}$$

wherein BSA is a body surface area of the patient and FusedCI is the combined cardiac index or the updated combined cardiac index.

Example 42. The system of any one of claims 26 to 41, wherein the offset is determined by the following equation:

$$B = \frac{( {S}_{FA} - A \times {S}_{FF} - {S}_{A} + A \times {S}_{F} )}{( {S}_{F} - M - β )}$$

wherein B is the offset, A is the scaling, β is a regularization parameter, where S_{F} is the temporally weighted APCO, S_{A} is the temporally weighted CCO, S_{FA} is the temporally weighted inner product of APCO and CCO, S_{FF} is the temporally weighted inner product of APCO with itself, and M is the temporally weighted counter.

Example 43. The system of claim 42, where β≥0.

Example 44. The system of claim 42, wherein S_{F}, S_{FF}, S_{FA}, S_{A}, and M, are computed for each thermodilution based cardiac output measurement as follows:

$${S}_{F} [ n ] = γ {S}_{F} [ n - 1 ] + ( 1 - γ ) \times {M}_{F} [ n ] ;$$

$${S}_{FF} [ n ] = γ {S}_{FF} [ n - 1 ] + ( 1 - γ ) \times {M}_{F} [ n ] \times {M}_{F} [ n ] ;$$

$${S}_{FA} [ n ] = γ {S}_{FA} [ n - 1 ] + ( 1 - γ ) \times {M}_{F} [ n ] \times {M}_{A} [ n ] ;$$

$${S}_{A} [ n ] = γ {S}_{A} [ n - 1 ] + ( 1 - γ ) \times {M}_{A} [ n ] ;$$

$$M [ n ] = γ M [ n - 1 ] + ( 1 - γ ) .$$

Example 45. The system of claim 44, wherein initial values are as follows: S_{F}=0, S_{FF}=0, S_{A}=0, S_{FA}=0, M=0, and γ is a forgetting factor that is >0 and <1.

Example 46. The system of any one of claims 26 to 45, wherein the one or more applications that can further direct the processor system to:

display the combined time varying cardiac output on an electronic visual display.

Example 47. The system of any one of claims 26 to 46, wherein the one or more applications that can further direct the processor system to:

provide an alert or fault if the combined computed cardiac index of the patient is above a high threshold or below a low threshold.

Example 48. The system of any one of claims 26 to 47, wherein the one or more applications that can further direct the processor system to:

provide an alert or fault if a trend the combined computed cardiac declines at a rate greater than a declination threshold or ascends at a rate greater than an ascension threshold.

Example 49. The system of any one of claims 26 to 48, wherein the peripheral arterial pressure sensor is a peripheral arterial line catheter.

Example 50. The system of any one of claims 26 to 48, wherein the peripheral arterial pressure sensor is a blood pressure cuff.

Example 51. A method of hemodynamic monitoring of a patient to provide a combined cardiac output measurement from two hemodynamic sensors, the method comprising:

sensing, using a peripheral arterial pressure sensor, a peripheral arterial pressure signal of a patient;
deriving, using a computational processing system and an arterial pressure-based cardiac output (APCO) algorithm, an APCO of the patient based upon the peripheral arterial pressure signal;
sensing, using a pulmonary artery catheter, a central blood flow signal of the patient,
deriving, using the computational processing system and a thermodilution based cardiac output algorithm, a thermodilution based cardiac output of the patient based upon the central blood flow signal;
deriving, using the computational processing system, a time-varying linear scaling equation, wherein the derivation of the time-varying linear scaling equation comprises:
setting a scaling factor to a preset value; and
determining an offset factor, wherein determining the offset factor comprises using the scaling factor, the temporally weighted APCO, and the temporally weighted thermodilution based cardiac output; and
   computing, using the computational processing system and the derived time-varying linear scaling equation, a combined cardiac output, wherein computing the combined cardiac output comprises applying a scaling factor and an offset factor to the APCO.

Example 52. The method of claim 51, wherein the APCO is iteratively derived at a first frequency and the thermodilution based cardiac output is iteratively derived at a second frequency.

Example 53. The method of claim 52, wherein the combined cardiac output is iteratively computed at the first frequency.

Example 54. The method of claim 52 or 53, wherein the scaling factor and the offset factor are each iteratively determined at the second frequency.

Example 55. The method of claim 52, 53, or 54, wherein the time-varying linear scaling equation is:

$${FusedCO}_{n 1} = {A}_{n 2} \times {APCO}_{n 1} + {B}_{n 2}$$

wherein n1 is the first frequency, n2 is the second frequency, A is the scaling factor, B is the offset factor, and FusedCO is the combined cardiac output.

Example 56. The method of any one of claims 52 to 55, wherein the first frequency is greater than the second frequency.

Example 57. The method of any one of claims 51 to 56, wherein the offset is determined by the following equation:

$$B = \frac{( {S}_{FA} - A \times {S}_{FF} - {S}_{A} + A \times {S}_{F} )}{( {S}_{F} - M - β )}$$

wherein B is the offset, A is the scaling, β is a regularization parameter, where S_{F} is the temporally weighted APCO, S_{A} is the temporally weighted CCO, S_{FA} is the temporally weighted inner product of APCO and CCO, S_{FF} is the temporally weighted inner product of APCO with itself, and M is the temporally weighted counter.

Example 58. The method of claim 57, where β≥0.

Example 59. The method of claim 57, wherein S_{F}, S_{FF}, S_{FA}, S_{A}, and M, are computed for each thermodilution based cardiac output measurement as follows:

$${S}_{F} [ n ] = γ {S}_{F} [ n - 1 ] + ( 1 - γ ) \times {M}_{F} [ n ] ;$$

$${S}_{FF} [ n ] = γ {S}_{FF} [ n - 1 ] + ( 1 - γ ) \times {M}_{F} [ n ] \times {M}_{F} [ n ] ;$$

$${S}_{FA} [ n ] = γ {S}_{FA} [ n - 1 ] + ( 1 - γ ) \times {M}_{F} [ n ] \times {M}_{A} [ n ] ;$$

$${S}_{A} [ n ] = γ {S}_{A} [ n - 1 ] + ( 1 - γ ) \times {M}_{A} [ n ] ;$$

$$M [ n ] = γ M [ n - 1 ] + ( 1 - γ ) .$$

Example 60. The method of claim 59, wherein initial values are as follows: S_{F}=0, S_{FF}=0, S_{A}=0, S_{FA}=0, M=0, and γ is a forgetting factor that is >0 and <1.

Example 61. The method of any one of claims 51 to 60 further comprising:

displaying the combined cardiac output on an electronic visual display.

Example 62. The method of any one of claims 51 to 61 further comprising:

providing an alert or fault if the combined cardiac output or a computed combined cardiac index of the patient is above a high threshold or below a low threshold.

Example 63. The method of any one of claims 51 to 62 further comprising:

providing an alert or fault if a trend the combined cardiac output or a computed combined cardiac index declines at a rate greater than a declination threshold or ascends at a rate greater than an ascension threshold.

Example 64. The method of any one of claims 51 to 63, wherein the peripheral arterial pressure sensor is a peripheral arterial line catheter.

Example 65. The method of any one of claims 51 to 63, wherein the peripheral arterial pressure sensor is a blood pressure cuff.

Example 66. A system for hemodynamic monitoring of a patient to provide a combined cardiac output measurement from two hemodynamic sensors, the system comprising:

a peripheral arterial pressure sensor for sensing a peripheral arterial pressure signal; a pulmonary artery catheter for sensing a central blood flow signal; and
a computational processing system in operable connection with the peripheral arterial pressure sensor and the pulmonary artery catheter; the computational processing system comprising:
a processor system; and
a memory system comprising one or more applications that can direct the processor system to:
   derive, using an arterial pressure-based cardiac output (APCO) algorithm, an APCO based upon the peripheral arterial pressure signal;
   derive, using a thermodilution based cardiac output algorithm, a thermodilution based cardiac output based upon the central blood flow signal;
   derive a time-varying linear scaling equation, wherein derivation of the time-varying linear scaling equation comprises:
      setting a scaling factor to a preset value; and
      determining an offset factor, wherein determining the offset factor comprises using the scaling factor, the temporally weighted APCO, and the temporally weighted thermodilution based cardiac output; and
   compute, using the derived time-varying linear scaling equation, a combined cardiac output, wherein computing the combined cardiac output comprises applying a scaling factor and an offset factor to the APCO.

Example 67. The system of claim 66, wherein the APCO is iteratively derived at a first frequency and the thermodilution based cardiac output is iteratively derived at a second frequency.

Example 68. The system of claim 67, wherein the combined cardiac output is iteratively computed at the first frequency.

Example 69. The system of claim 67 or 68, wherein the scaling factor and the offset factor are each iteratively determined at the second frequency.

Example 70. The system of claim 67, 68, or 69, wherein the time-varying linear scaling equation is:

$${FusedCO}_{n 1} = {A}_{n 2} \times {APCO}_{n 1} + {B}_{n 2}$$

wherein n1 is the first frequency, n2 is the second frequency, A is the scaling factor, B is the offset factor, and FusedCO is the combined cardiac output.

71. The system of any one of claims 67 to 70, wherein the first frequency is greater than the second frequency.

72. The system of any one of claims 66 to 71, wherein the offset is determined by the following equation:

$$B = \frac{( {S}_{FA} - A \times {S}_{FF} - {S}_{A} + A \times {S}_{FF} )}{( {S}_{F} - M - β )}$$

wherein B is the offset, A is the scaling, β is a regularization parameter, where S_{F} is the temporally weighted APCO, S_{A} is the temporally weighted CCO, S_{FA} is the temporally weighted inner product of APCO and CCO, S_{FF} is the temporally weighted inner product of APCO with itself, and M is the temporally weighted counter.

Example 73. The system of claim 72, where β≥0.

Example 74. The system of claim 72, wherein S_{F}, S_{FF}, S_{FA}, S_{A}, and M, are computed for each thermodilution based cardiac output measurement as follows:

$${S}_{F} [ n ] = γ {S}_{F} [ n - 1 ] + ( 1 - γ ) \times {M}_{F} [ n ] ;$$

$${S}_{FF} [ n ] = γ {S}_{FF} [ n - 1 ] + ( 1 - γ ) \times {M}_{F} [ n ] \times {M}_{F} [ n ] ;$$

$${S}_{FA} [ n ] = γ {S}_{FA} [ n - 1 ] + ( 1 - γ ) \times {M}_{F} [ n ] \times {M}_{A} [ n ] ;$$

$${S}_{A} [ n ] = γ {S}_{A} [ n - 1 ] + ( 1 - γ ) \times {M}_{A} [ n ] ;$$

$$M [ n ] = γ M [ n - 1 ] + ( 1 - γ ) .$$

Example 75. The system of claim 74, wherein initial values are as follows: S_{F}=0, S_{FF}=0, S_{A}=0, S_{FA}=0, M=0, and γ is a forgetting factor that is >0 and <1.

Example 76. The system of any one of claims 66 to 75, wherein the one or more applications that can further direct the processor system to:

display the combined cardiac output or a computed combined cardiac index on an electronic visual display.

Example 77. The system of any one of claims 66 to 76, wherein the one or more applications that can further direct the processor system to:

provide an alert or fault if the combined cardiac output or a computed combined cardiac index of the patient is above a high threshold or below a low threshold.

Example 78. The system of any one of claims 66 to 77, wherein the one or more applications that can further direct the processor system to:

provide an alert or fault if the combined cardiac output or a computed combined cardiac index declines at a rate greater than a declination threshold or ascends at a rate greater than an ascension threshold.

Example 79. The system of any one of claims 66 to 78, wherein the peripheral arterial pressure sensor is a peripheral arterial line catheter.

Example 80. The system of any one of claims 66 to 78, wherein the peripheral arterial pressure sensor is a blood pressure cuff.

1. A method of hemodynamic monitoring of a patient to provide a combined cardiac output measurement from two hemodynamic sensors, the method comprising:
sensing, using a peripheral arterial pressure sensor, a peripheral arterial pressure signal of a patient;
deriving, using a computational processing system and an arterial pressure-based cardiac output (APCO) algorithm, an APCO of the patient based upon the peripheral arterial pressure signal;
sensing, using a central artery catheter sensor, a central blood flow signal of the patient,
deriving, using the computational processing system and a thermodilution based cardiac output algorithm, a thermodilution based cardiac output of the patient based upon the central blood flow signal;
deriving, using the computational processing system, a time-varying linear scaling equation,
wherein derivation of the time-varying linear scaling equation comprises:
determining a scaling factor and an offset factor;
wherein determining the scaling factor comprises determining whether the scaling factor is a preset value or a computed value using a temporally weighted APCO and a temporally weighted thermodilution based cardiac output; and
wherein determining the offset factor comprises using the scaling factor, the temporally weighted APCO, and the temporally weighted thermodilution based cardiac output; and
computing, using the computational processing system and the derived time-varying linear scaling equation, a combined cardiac output, wherein computing the combined cardiac output comprises applying the scaling factor and the offset factor to the APCO.

2. The method of claim 1, wherein the APCO is iteratively derived at a first frequency and the thermodilution based cardiac output is iteratively derived at a second frequency.

3. The method of claim 2, wherein the combined cardiac output is iteratively computed at the first frequency.

4. The method of claim 2, wherein the scaling factor and the offset factor are each iteratively determined at the second frequency.

5. The method of claim 2, wherein the time-varying linear scaling equation is:
$${FusedCO}_{n 1} = {A}_{n 2} \times {APCO}_{n 1} + {B}_{n 2}$$
wherein n1 is the first frequency, n2 is the second frequency, A is the scaling factor, B is the offset factor, and FusedCO is the combined cardiac output.

6. The method of claim 2, wherein the first frequency is greater than the second frequency.

7. The method of claim 1, wherein the scaling factor is determined to be the preset value when the difference between the temporally weighted APCO and the temporally weighted thermodilution based cardiac output is less than a first cardiac output threshold and a first combined cardiac index of the patient is greater than a cardiac index threshold.

8. The method of claim 7, wherein the scaling factor is determined to be the computed value when the difference between the temporally weighted APCO and the temporally weighted thermodilution based cardiac output is greater than the first cardiac output threshold.

9. The method of claim 8, wherein when the difference between the temporally weighted APCO and the temporally weighted thermodilution based cardiac output is greater than the first cardiac output threshold and less than a second cardiac output threshold, the computed value of the scaling factor is computed as follows:
$${A}_{reduced} = {A}_{preset} \times [ \frac{{S}_{A}}{{S}_{F}} \times ( {CO}_{diff} - {CO}_{lowThresh} ) + ( {CO}_{highThresh} - {CO}_{diff} ) ]$$
wherein A_{reduced} is the computed value of the scaling factor; Aₚᵣₑₛₑₜ is the preset value of the scaling factor; S_{F} is the temporally weighted APCO; S_{A} is the temporally weighted thermodilution based cardiac output; COthresh1 is the first cardiac output threshold; COthresh2 is the second cardiac output threshold; COthresh1<COthresh2; and
CO_{diff}=S_{F}−S_{A}.

10. The method of claim 2, wherein when the difference between the temporally weighted APCO and the temporally weighted thermodilution based cardiac output (S_{A}) is greater than or equal to the second cardiac output threshold, the computed value of the scaling factor is computed as follows:
$${A}_{reduced} = {A}_{preset} \times \frac{{S}_{A}}{{S}_{F}}$$
wherein A_{reduced} is the computed value of the scaling factor; Aₚᵣₑₛₑₜ is the preset value of the scaling factor; S_{F} is the temporally weighted APCO and S_{A} is the temporally weighted thermodilution based cardiac output.

11. The method of claim 7, wherein the scaling factor is determined to be the computed value when the first combined cardiac index of the patient is less than the cardiac index threshold; and wherein when the first combined cardiac index of the patient is less than the cardiac index threshold, the computed value of the scaling factor is computed by reducing the preset scaling factor by a first numerical factor.

12. The method of claim 1, wherein the offset is determined by the following equation:
$$B = \frac{( {S}_{FA} - A \times {S}_{FF} - {S}_{A} + A \times {S}_{F} )}{{〚 ( S 〛}_{F} - M - β )}$$
wherein B is the offset, A is the scaling, β is a regularization parameter, where S_{F} is the temporally weighted APCO, S_{A} is a temporally weighted CCO, S_{FA} is a temporally weighted inner product of APCO and CCO, S_{FF} is a temporally weighted inner product of APCO with itself, and Mis a temporally weighted counter.

13. The method of claim 12, wherein β≥0.

14. The method of claim 12, wherein S_{F}, SFF, SFA, S_{A}, and M, are computed for each thermodilution based cardiac output measurement as follows:
$${S}_{F} [ n ] = γ {S}_{F} [ n - 1 ] + ( 1 - γ ) \times {M}_{F} [ n ] ;$$
$${S}_{FF} [ n ] = γ {S}_{FF} [ n - 1 ] + ( 1 - γ ) \times {M}_{F} [ n ] \times {M}_{F} [ n ] ;$$
$${S}_{FA} [ n ] = γ {S}_{FA} [ n - 1 ] + ( 1 - γ ) \times {M}_{F} [ n ] \times {M}_{A} [ n ] ;$$
$${S}_{A} [ n ] = γ {S}_{A} [ n - 1 ] + ( 1 - γ ) \times {M}_{A} [ n ] ;$$
$$M [ n ] = γ M [ n - 1 ] + ( 1 - γ ) .$$

15. The method of claim 1, wherein the peripheral arterial pressure sensor is a peripheral arterial line catheter or a blood pressure cuff, wherein the central artery catheter sensor is a pulmonary artery catheter.

16. A system for hemodynamic monitoring of a patient to provide a combined cardiac output measurement from two hemodynamic sensors, the system comprising:
a peripheral arterial pressure sensor for sensing a peripheral arterial pressure signal;
a central artery catheter sensor for sensing a central blood flow signal; and
a computational processing system in operable connection with the peripheral arterial pressure sensor and the pulmonary artery catheter, the computational processing system comprising:
a processor system; and
a memory system comprising one or more applications that can direct the processor system to:
derive, using an arterial pressure-based cardiac output (APCO) algorithm, an APCO based upon the peripheral arterial pressure signal;
derive, using a thermodilution based cardiac output algorithm, a thermodilution based cardiac output based upon the central blood flow signal;
derive a time-varying linear scaling equation, wherein derivation of the time-varying linear scaling equation comprises:
determining a scaling factor and an offset factor, wherein determining the scaling factor comprises determining whether the scaling factor is a preset value or a computed value using a temporally weighted APCO and a temporally weighted thermodilution based cardiac output, and wherein determining the offset factor comprises using the scaling factor, the temporally weighted APCO, and the temporally weighted thermodilution based cardiac output; and
compute, using the derived time-varying linear scaling equation, a combined cardiac output, wherein computing the combined cardiac output comprises applying a scaling factor and an offset factor to the APCO.

17. The system of claim 16, wherein the APCO is iteratively derived at a first frequency and the thermodilution based cardiac output is iteratively derived at a second frequency.

18. The system of claim 17, wherein the combined cardiac output is iteratively computed at the first frequency.

19. The system of claim 17, wherein the scaling factor and the offset factor are each iteratively determined at the second frequency.

20. The system of claim 17, wherein the time-varying linear scaling equation is:
$${FusedCO}_{n 1} = {A}_{n 2} \times {APCO}_{n 1} + {B}_{n 2}$$
wherein n1 is the first frequency, n2 is the second frequency, A is the scaling factor, B is the offset factor, and FusedCO is the combined cardiac output.

21. The system of claim 17, wherein the first frequency is greater than the second frequency.

22. The system of claim 16, wherein the scaling factor is determined to be the preset value when the difference between the temporally weighted APCO and the temporally weighted thermodilution based cardiac output is less than a first cardiac output threshold and a first combined cardiac index of the patient is greater than a cardiac index threshold.

23. The system of claim 22, wherein the scaling factor is determined to be the computed value when the difference between the temporally weighted APCO and the temporally weighted thermodilution based cardiac output is greater than the first cardiac output threshold.

24. The system of claim 23, wherein when the difference between the temporally weighted APCO and the temporally weighted thermodilution based cardiac output is greater than the first cardiac output threshold and less than a second cardiac output threshold, the computed value of the scaling factor is computed as follows:
$${A}_{reduced} = {A}_{preset} \times [ \frac{{S}_{A}}{{S}_{F}} \times ( {CO}_{diff} - {CO}_{lowThresh} ) + ( {CO}_{highThresh} - {CO}_{diff} ) ]$$
wherein A_{reduced} is the computed value of the scaling factor; Aₚᵣₑₛₑₜ is the preset value of the scaling factor; S_{F} is the temporally weighted APCO; S_{A} is the temporally weighted thermodilution based cardiac output; COthresh1 is the first cardiac output threshold; COthresh2 is the second cardiac output threshold; COthresh1<COthresh2; and
CO_{diff}=S_{F}−S_{A}.

25. The system of claim 23, wherein when the difference between the temporally weighted APCO and the temporally weighted thermodilution based cardiac output (S_{A}) is greater than or equal to the second cardiac output threshold, the computed value of the scaling factor is computed as follows:
$${A}_{reduced} = {A}_{preset} \times \frac{{S}_{A}}{{S}_{F}}$$
wherein A_{reduced} is the computed value of the scaling factor; Aₚᵣₑₛₑₜ is the preset value of the scaling factor; S_{F} is the temporally weighted APCO and S_{A} is the temporally weighted thermodilution based cardiac output.

26. The system of claim 22, wherein the scaling factor is determined to be the computed value when the first combined cardiac index of the patient is less than the cardiac index threshold; and wherein when the first combined cardiac index of the patient is less than the cardiac index threshold, the computed value of the scaling factor is computed by reducing the preset scaling factor by a first numerical factor.

27. The system of claim 16, wherein the offset is determined by the following equation:
$$B = \frac{( {S}_{FA} - A \times {S}_{FF} - {S}_{A} + A \times {S}_{F} )}{{〚 ( S 〛}_{F} - M - β )}$$
wherein B is the offset, A is the scaling, β is a regularization parameter, where S_{F} is the temporally weighted APCO, S_{A} is a temporally weighted CCO, S_{FA} is a temporally weighted inner product of APCO and CCO, S_{FF} is a temporally weighted inner product of APCO with itself, and M is a temporally weighted counter.

28. The system of claim 27, where β≥0.

29. The system of claim 27, wherein S_{F}, S_{FF}, S_{FA}, S_{A}, and M, are computed for each thermodilution based cardiac output measurement as follows:
$${S}_{F} [ n ] = γ {S}_{F} [ n - 1 ] + ( 1 - γ ) \times {M}_{F} [ n ] ;$$
$${S}_{FF} [ n ] = γ {S}_{FF} [ n - 1 ] + ( 1 - γ ) \times {M}_{F} [ n ] \times {M}_{F} [ n ] ;$$
$${S}_{FA} [ n ] = γ {S}_{FA} [ n - 1 ] + ( 1 - γ ) \times {M}_{F} [ n ] \times {M}_{A} [ n ] ;$$
$${S}_{A} [ n ] = γ {S}_{A} [ n - 1 ] + ( 1 - γ ) \times {M}_{A} [ n ] ;$$
$$M [ n ] = γ M [ n - 1 ] + ( 1 - γ ) .$$

30. The system of claim 16, wherein the peripheral arterial pressure sensor is a peripheral arterial line catheter or a blood pressure cuff, wherein the central artery catheter sensor is a pulmonary artery catheter.
